(19) United States
(12) Patent Application Publication
(10) Pub. No.: US 2023/0113183 A1
(43) Pub. Date: Apr. 13, 2023

(54) CELL

(71) Applicant: AUTOLUS LIMITED, London (GB)

(72) Inventors: Callum McKenzie, London (GB); Shaun Cordoba, London (GB); MArtin Pulé, London (GB)

(21) Appl. No.: 17/915,637

(22) PCT Filed: Apr. 8, 2021
(86) PCT No.: PCT/GB2021/050866

§ 371 Date: Sep. 29, 2022

(30) Foreign Application Priority Data
Apr. 9, 2020 (GB) 2005331.0
Nov. 2, 2020 (GB) 2017358.9

(51) Int. Cl. C12N 5/0783; C07K 14/705; C07K 14/725; C12N 15/63

(52) U.S. Cl. CPC C12N 5/0636; C07K 14/70578; C07K 14/7051; C12N 15/63; C07K 2319/03

(57) ABSTRACT

The present invention relates to a cell which comprises; (a) a chimeric antigen receptor (CAR) or a transgenic T-cell receptor (TCR); and (b) a FasL-binding receptor (FLBR) comprising; (i) a Fas ectodomain and a TNFR endodomain, wherein the TNFR endodomain comprises the signalling portion of the decoy receptor 2 (DcR2), GITR, CD30, XEDAR, CD40, CD27, BCMA or Fn14 endodomain, or (ii) a membrane-bound decoy receptor 3 (DcR3).

### FIELD OF THE INVENTION

The present invention relates to an engineered cell which expresses a chimeric antigen receptor (CAR) or a transgenic T-cell receptor (TCR); and a Fas ligand (FasL)-binding receptor.

### BACKGROUND TO THE INVENTION

The tumour microenvironment (TME) provides tumour cells with essential signals for survival, growth and immune resistance. The TME is immunosuppressive and can inhibit the persistence and survival of immune cells in cancer immunotherapies, such as CAR T cell therapy. Immunosuppressive mechanisms employed by the TME include e.g., upregulating immune checkpoint signals such as PDL-1 and/or CTLA-4 and secretion of cytokines such as IL-6 and/or TGF-beta.

Recent evidence suggests that the immunosuppressive TME also upregulates death ligands such as Fas ligand (FasL). FasL induces apoptosis of immune cells that express death receptors for Fas, such as tumour-infiltrating lymphocytes (TILs).

In addition to the TME upregulating death ligands, it has also been shown that CAR-T cells themselves upregulate death receptors and their ligands upon activation and transduction of the CAR construct, triggering activation-induced death (AICD) and further exacerbating the problem of CAR-T cell persistence in the TME. Moreover, FasL is not only expressed by activated T cells but is also upregulated by exposure to IFNγ that is produced by activated T cells.

Notably, third generation CARs, which have two co-stimulatory endodomains, seem to be particularly susceptible to AICD as a result of increased FasL expression (Xu et al., 2017, Hum Vaccin Immunother. 13(7):1548-1555, Benmebarek et al., 2019, In J Mol Sci. 20(6): 1283).

Scientists have attempted to block the Fas/FasL interaction with antibody technology, but in vivo data showed a significant amount of antibody was needed every two to three days causing significant side-effects in practice (Ma et al., 2016, PLoS Pathog. 12(5): e1005642).

Furthermore, blocking FasL with an antibody could hinder the killing effects of the CAR T cell since FasL is an important weapon for T cells to exert killing (Gargett et al., 2016, Mol Ther. 24:1135-49).

Accordingly, there is a need for an alternative approach to abrogate death-receptor stimulation on CAR T cells to alleviate this immune checkpoint and improve the effectiveness of engineered cells to persist and survive in the TME.

### SUMMARY OF THE INVENTION

The present inventors have determined that a cell expressing a receptor which competitively binds to FasL and neutralises the Fas-FasL pathway, can reduce or prevent Fas-induced apoptosis. Co-expression of such a receptor with a CAR or transgenic TCR and improves infiltration of the CAR/TCR expressing cell into tumour vasculature and its persistence within the TME.

The inventors propose two alternative approaches to achieve neutralisation of the Fas-FasL pathway, both of which competitively bind to FasL, blocking its capacity to trigger trimerization and recruit a protein called Fas-associated death domain (FADD) via homotypic interactions of their respective death domains.

The first approach involves fusing the extracellular domain of Fas receptor to a Tumour Necrosis Factor Receptor (TNFR) intracellular signalling domain. The second approach involves expressing a membrane-bound decoy receptor 3 (DcR3) which binds to FasL.

Accordingly, in one aspect, the present invention provides a cell which comprises (a) a chimeric antigen receptor (CAR) or a transgenic T-cell receptor (TCR); and (b) a FasL-binding receptor (FLBR) comprising; (i) a Fas ectodomain and a TNFR endodomain, wherein the TNFR endodomain comprises the signalling portion of the decoy receptor 2 (DcR2), GITR, CD30, XEDAR, CD40, CD27, BCMA or Fn14 endodomain, or (ii) a membrane-bound decoy receptor 3 (DcR3).

Unlike, the endodomain of the Fas receptor, the TNFR endodomains of the present invention are not able to bind via homotypic interactions to the death domains on Fas-associated death domain (FADD), and therefore assembly of the death inducing signalling complex (DISC) is inhibited, which ultimately inhibits the Fas-FasL pathway to apoptosis. Similarly, membrane-bound DcR3 out-competes endogenous Fas receptor for binding to FasL, neutralising the Fas-FasL pathway.

In the first approach described above, the FLBR of the invention may have the general structure:

Fas exo-TM-TNFR endo

in which:
Fas exo is Fas ectodomain;
TM is a transmembrane domain; and
TNFR endo is a TNFR endodomain.

In particular the FLBR may comprise a Fas ectodomain and a CD40 endodomain. The CD40 endodomain may comprise the sequence shown as SEQ ID No. 4.

The FLBR of the present invention may comprise a Fas transmembrane domain. Alternatively, the transmembrane domain of the FLBR may comprise a CD28 transmembrane domain, a CD8a transmembrane domain, a DcR2 transmembrane domain, a TYRP-1 transmembrane domain or an EGFR transmembrane domain.

In the second approach described above, the FLBR may have the general structure:

DcR3-spacer-TM-endo

in which:

DcR3 comprises the FasL binding domain of DcR3

spacer is a spacer sequence connecting DcR3 to the transmembrane domain

TM is a transmembrane domain

endo is an optional intracellular sequence.

The membrane-bound DcR3 may be tethered to the membrane via a CD8 transmembrane stalk. Alternatively, the membrane-bound DcR3 may be tethered to the membrane via any sequence which capable of tethering the otherwise soluble decoy receptor to the plasma membrane.

The membrane-bound DcR3 may comprise mutations at the C-terminal Heparin Binding Domain (HBD). This is because the HBD binds to heparan sulphate proteoglycans (HSPG) which via crosslinking can induce apoptosis in dendritic cells. More specifically, the HBD C-terminal portion of the membrane-bound DcR3 may comprise have mutations at one or more of positions K256, R258 and R259, with reference to the sequence shown as SEQ ID No. 9 below. Mutating these otherwise basic amino acids to alanine residues abolishes binding to HSPGs.

The FLBR may comprise a sequence selected from Fas-DcR2 (SEQ ID NO: 35), Fas-GITR (SEQ ID NO: 36), Fas-CD30 (SEQ ID NO: 37), Fas-XEDAR (SEQ ID NO: 38), Fas-CD27 (SEQ ID NO: 39), Fas-BCMA (SEQ ID NO: 40), Fas-CD40 (SEQ ID NO: 41), Fas-Fn14 (SEQ ID NO: 42), DcR3-CD8STK (SEQ ID NO: 43) and DcR3mut-CD8STK (SEQ ID NO: 44) or a variant with at least 80% sequence identity to any of SEQ ID NO: 35-44. The FLBR may also comprise more than one TNFR endodomain. For example, the sequence of the FLBR may be Fas-GITR-GITR, Fas-GITR-CD30, Fas-GITR-XEDAR or Fas-GITR-DcR2. Alternatively, the FLBR may be, for example, DcR3-41BB, DcR3-OX40, DcR3-XEDAR, DcR3-GITR, DcR3-CD40, DcR3-CD27, DcR3-BCMA or DcR3-Fn14.

In a second aspect, the invention provides a FasL-binding receptor (FLBR) comprising a Fas ectodomain and a TNFR endodomain, wherein the TNFR endodomain comprises the signalling portion of the decoy receptor 2 (DcR2), GITR, CD30, XEDAR, CD40, CD27, BCMA or Fn14 endodomain. In particular, the TNFR endodomain may be a CD40 endodomain.

In a third aspect, the invention provides a nucleic acid sequence which encodes the FLBR of the present invention.

In a fourth aspect, the present invention provides a nucleic acid construct which comprises: (a) a first nucleic acid sequence which encodes a chimeric antigen receptor (CAR) or a transgenic T-cell receptor (TCR); and (b) a second nucleic acid sequence which encodes a FasL-binding receptor (FLBR) as defined above.

The first and second nucleic acid sequences may be separated by a co-expression site, such as a sequence encoding a self-cleaving peptide.

In a fifth aspect, the present invention provides a kit of nucleic acid sequences comprising: (a) a first nucleic acid sequence which encodes a chimeric antigen receptor (CAR) or a transgenic T-cell receptor (TCR); and (b) a second nucleic acid sequence which encodes a FasL-binding receptor (FLBR) as defined above.

In a sixth aspect, the present invention provides a vector which comprises a nucleic acid sequence according to the third aspect of the invention or a nucleic acid construct according to the fourth aspect of the invention.

In a seventh aspect, the present invention provides a kit of vectors which comprises: (a) a first vector which comprises a nucleic acid sequence which encodes a chimeric antigen receptor (CAR) or a transgenic T-cell receptor (TCR); and

(b) a second vector which comprises a nucleic acid sequence which encodes a FasL-binding receptor (FLBR) as defined above.

In an eighth aspect, the present invention provides a pharmaceutical composition which comprises a plurality of cells according to the first aspect of the present invention.

In a ninth aspect the invention provides the pharmaceutical composition according to the eighth aspect of the invention for use in treating and/or preventing a disease.

In a tenth aspect, the present invention provides a method for treating and/or preventing a disease, which comprises the step of administering a pharmaceutical composition according to the eighth aspect of the invention to a subject in need thereof.

The method may comprise the following steps: (i) isolation of a cell containing sample; (ii) transduction or transfection of the cell a nucleic acid sequence according to the third aspect of the invention, a nucleic acid construct according to the fourth aspect of the invention, a kit of nucleic acid sequences according to the fifth aspect of the invention; a vector according to the sixth aspect of the invention; or a kit of vectors according to the seventh aspect of the invention; and (iii) administering the cells from (ii) to a subject.

The cell may autologous or allogenic.

In an eleventh aspect, the present invention provides use of a pharmaceutical composition according eighth aspect of the invention in the manufacture of a medicament for the treatment and/or prevention of a disease.

The disease is may be cancer.

In a twelfth aspect, the present invention provides a method for making a cell according to the first aspect of the invention, which comprises the step of introducing: a nucleic acid sequence according to the third aspect of the invention, a nucleic acid construct according to the fourth aspect of the invention, a kit of nucleic acid sequences according to the fifth aspect of the invention; a vector according to the sixth aspect of the invention; or a kit of vectors according to the seventh aspect of the invention into the cell in vitro.

The cell may be from a sample isolated from a subject.

Surprisingly, the inventors have found the expression of a FasL-binding receptor (FLBR) of the present invention by a cell conveys upon that cell greater resistance to FasL-mediated apoptosis than a cell expressing a Fas receptor with simply a truncated Fas intracellular death domain. The inventors propose to exploit this finding to generate CAR- and TCR-expressing cells with more effective resistance to FasL-induced within the TME, thus providing a superior immunotherapy.

### BRIEF DESCRIPTION OF THE FIGURES

FIG. 1—(a) Schematic diagram illustrating a classical CAR. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in the same compound endodomain.

FIG. 2—Schematic diagram illustrating WT Fas-FasL-inducing apoptosis (a) and Fas-L binding Receptors (FLBRs): Fas-DcR2 (b), Fas-TNFR (c), Membrane-bound DcR3 (d) and structure of DcR3 (e).

FIG. 3—Summary of the TNF superfamily. Both Fas and TRAIL receptor molecules use FADD as an adaptor molecule and TNFR1 and D3 use TRADD as an adaptor molecule.

FIG. 4—Schematic of Fas-FasL interactions in the tumour microenvironment (TME). FasL is expressed by T-cell/CAR-T cells and overactivated T cells leading to activation-induced cell death (AICD) (a), MDSC (b), T regs (c), tumour-endothelial cells (d), cancer cells (e) and cancer-secreting exosomes (f).

FIG. 5—Schematic diagram of T cell co-culture assays.

FIG. 6—Flow cytometry data of total cell survival count comparing CARs co-expressed with FasL-binding constructs, made relative to cell count from Fmc63 (antiCD19 CAR) transduced PBMCs. The FasL-binding receptor (FLBR) constructs Fas-DcR2, Fas-GITR and Fas-CD30 and DcR3-CD8stk show superior cell death rescuing over the other constructs.

FIG. 7—Flow cytometry data comparing total cell survival count of a CAR co-expressed with FasL-binding constructs DcR3 and DcR3-CD8stk. Membrane-bound DcR3-CD8stk showed superior cell survival over soluble DcR3.

FIG. 8—Flow cytometry data comparing survival of transduced RQR8 positive cells. The FasL-binding receptor (FLBR) constructs Fas-DcR2, Fas-GITR and Fas-CD30 show superior cell death rescuing over the other constructs. The dotted line indicates the average survival of PBMCs transduced to coexpress Fmc63, FasΔDD and FasL.

FIG. 9—Flow cytometry data of total cell survival count using immobilised recombinant soluble Fas ligand to induce cell death, comparing CAR co-expressed with FasL-binding constructs, made relative to Fmc63. The FasL-binding receptor (FLBR) constructs Fas-DcR2 and Fas-GITR show superior cell death rescuing over the other constructs.

FIG. 10—Flow cytometry data of transduced RQR8 positive cell survival using immobilised recombinant soluble Fas ligand to induce cell death comparing CAR co-expressed with FasL binding constructs relative to Fmc63.

FIG. 11—Flow cytometry data using immobilised soluble recombinant Fas ligand to induce cell death comparing survival of absolute RQR8 positive cell count. The FasL-binding receptor construct Fas-XEDAR constitutively proliferates over the five day time course and rescues FasL induced cell death.

FIG. 12—Flow cytometry data using immobilised soluble Fas ligand to induce cell death comparing proliferation fold difference of absolute RQR8 positive cell count between day 5 and day 0. The FasL-binding receptor construct Fas-XEDAR shows a much higher proliferation fold difference than the truncated Fas intracellular death domain construct.

FIG. 13—Flow cytometry data using immobilised soluble Fas ligand to induce cell death comparing the percentage of transduced RQR8 positive cells. The FasL-binding receptor (FLBR) construct Fas-XEDAR shows a higher enrichment of RQR8 positive cells over the truncated Fas intracellular death domain at day 0 and at day 5.

FIG. 14—Flow cytometry data using immobilised soluble Fas ligand to induce cell death comparing survival of transduced RQR8 positive cells. The FasL-binding receptor (FLBR) construct Fas-XEDAR shows protection from FasL-mediated cell death, apparent under “FasL+anti-Fmc63” condition.

FIG. 15—Flow cytometry data using immobilised soluble Fas ligand to induce cell death showing the FasL binding receptor (FLBR) construct Fas-XEDAR secretes a significantly higher basal interferon gamma than truncated Fas intracellular death domain.

FIG. 16—Flow cytometry data showing the FasL binding receptor (FLBR) construct Fas-XEDAR secretes significantly higher inducible IL-2 than truncated Fas intracellular death domain.

FIG. 17—Flow cytometry data using immobilised soluble Fas ligand to induce cell death comparing survival and absolute cell counts of transduced RQR8 positive cells. Surprisingly, PBMCs transduced with the FLBR constructs Fas-CD27, Fas-CD40, Fas-BCMA and Fas-Fn14 have higher absolute RQR8 positive cell counts compared to FasΔDD and Fas-41 BB.

FIG. 18—Flow cytometry data using immobilised soluble Fas ligand to induce cell death comparing absolute cell counts of transduced RQR8 positive cells made relative to transduced PBMCs incubated on PBS-treated wells. PBMCs transduced with FLBR constructs Fas-CD27, Fas-CD40, Fas-BCMA and Fas-Fn14 have superior proliferation when incubated with immobilised FasL compared to FasΔDD.

FIG. 19—Flow cytometry data using immobilised soluble Fas ligand to induce cell death comparing the proliferation fold difference of transduced RQR8 positive cells (absolute cell count from day 5 versus absolute cell count from day 0). PBMCs transduced with FLBR constructs Fas-CD27, Fas-CD40, Fas-BCMA and Fas-Fn14 have superior proliferation when incubated with immobilised FasL compared to FasΔDD and Fas-41BB. FLBR constructs Fas-CD40, Fas-BCMA and Fas-Fn14 also induce constitutive proliferation (comparing the PBS condition).

FIG. 20—Flow cytometry data using immobilised soluble Fas ligand to induce cell death showing FLBR constructs Fas-CD27, Fas-CD40, Fas-BCMA and Fas-Fn14 secretes significantly higher interferon gamma than FasΔDD when incubated with Fas ligand. FLBR constructs Fas-CD40, Fas-BCMA and Fas-Fn14 also induces basal interferon gamma secretion (comparing the PBS condition).

FIG. 21—Fas-XEDAR, Fas-CD40, Fas-BCMA and Fas-Fn14 co-expressed with a GD2-targeting CAR increases cytotoxic potential after repeated encounters with antigen. GD2 targeting CAR T-cells were co-cultured with SupT1 GD2 targets at a 1:1 effector to target ratio. Every 3 or 4 days, CAR T-cells were re-stimulated with 0.5×10⁵ SupT1 GD2 cells/well. Target cell killing was quantified by FACS before each new re-stimulation. Remaining viable target cells were defined by their exclusion of Sytox Blue and the absence of CD2 and CD3 expression, while T-cells were defined by the expression of CD2 and CD3. Lines represent the median value of 3 separate PBMC donors.

### DETAILED DESCRIPTION OF THE INVENTION

Fas-FasL Pathway

The Fas receptor (CD95, tumour necrosis factor receptor superfamily member 6, Uniprot ID: P25445) is a type 1 transmembrane glycoprotein receptor of relative molecular mass ˜45,000 which is localized on the surface of various cells including lymphocytes and hepatocytes. The Fas receptor triggers a signal transduction pathway leading to apoptosis and expression of Fas can be increased by activation of lymphocytes as well as by cytokines such as IFNγ and TNF. The interaction of Fas with its ligand FasL (FasL/CD95L, Uniprot ID: P48023) regulates numerous physiological and pathological processes that are mediated through programmed cell death.

Both Fas and FasL are members of the TNF-R superfamily, which contain one to five extracellular cysteine rich domains (CRDs) and, in their cytoplasmic tail, a death domain (DD), which is made up of 80-100 residue long motifs.

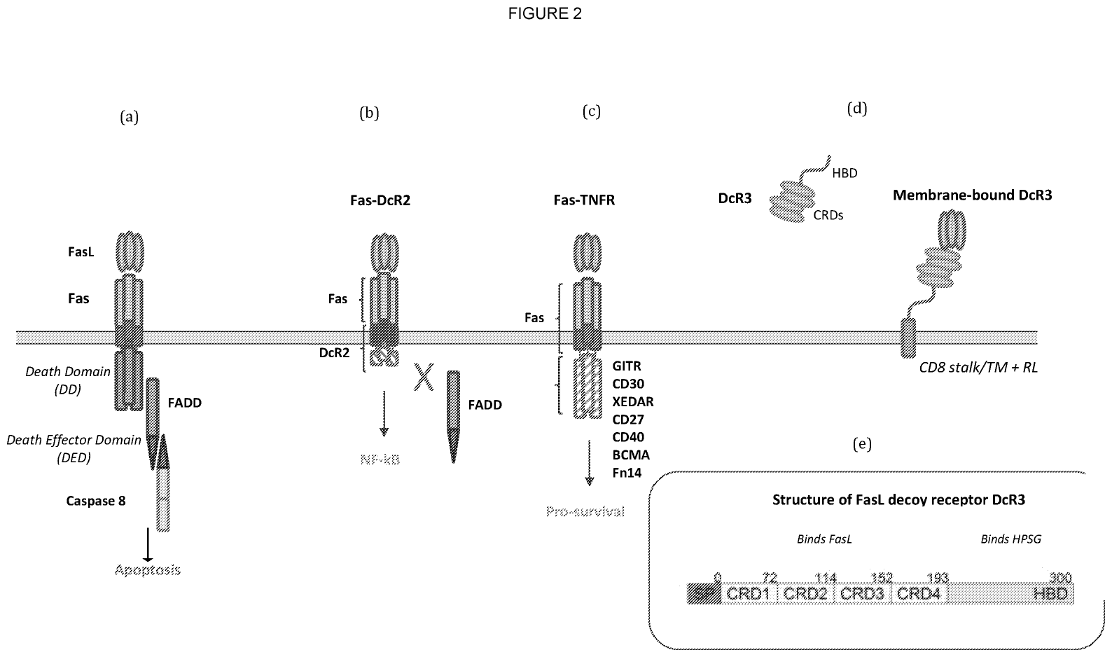

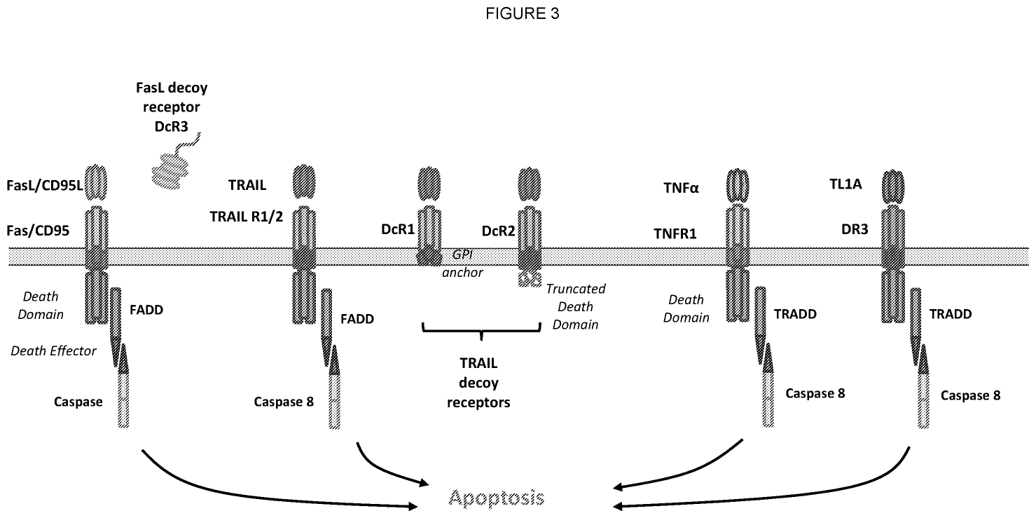

Fas binding to FasL triggers receptor trimerization and recruitment of a protein called Fas-associated death domain (FADD) via homotypic interactions of their death domains (DDs). In turn, FADD then recruits procaspase-8 to the activated receptor and the resulting death-inducing signaling complex (DISC) performs caspase-8 proteolytic activation, which initiates the subsequent cascade of caspases (aspartate-specific cysteine proteases) mediating apoptosis (FIGS. 2a and 3).

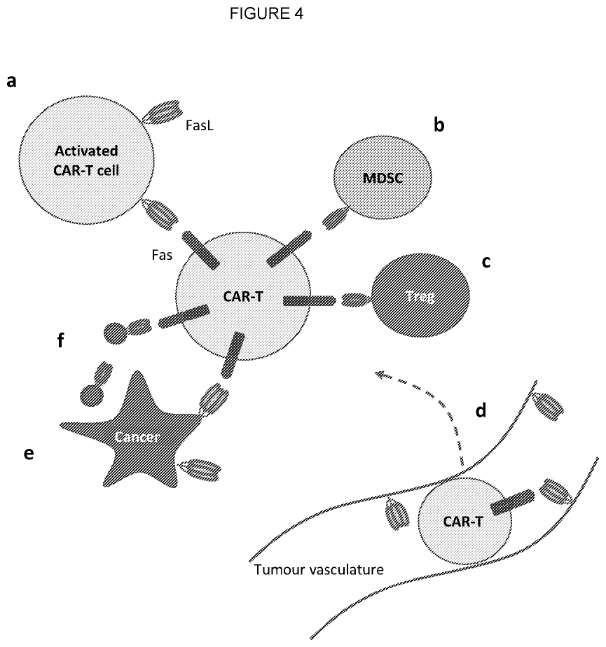

FasL is an important immune checkpoint as it is overexpressed by many cells within the TME (FIG. 4). FasL has been reported to be expressed by many cancers themselves such as melanomas, lung carcinoms, hepatocellular carcinomas, esophageal carcinomas and colon carcinomas.

In addition, tumour endothelial cells, which line blood vessels and control blood and nutrient flow and trafficking of leukocytes, have been shown to express FasL, whereas normal vasculature do not.

Furthermore, FasL is expressed by myeloid-derived suppressor cells (MDSC). MDSC are a heterogenous populations of cells that expand during cancer, chronic inflammation, autoimmune and infectious diseases, and dampen down the immune response thereby promoting tumour growth. Finally, FasL is also reportedly expressed by cancer-associated fibroblasts (CAFs) and CD4+CD25+ regulatory T cells.

FasL is also expressed by T cells and has been shown to be further upregulated in CAR-Ts, meaning CAR T cells are susceptible to fratricide. Finally, FasL is not only expressed by activated T cells but is also upregulated by exposure to IFNγ that is produced by activated T cells.

As a mechanism of T-cell homeostasis, T cells that are constantly activated die through a mechanism called activation-induced cell death (AICD), and the Fas-FasL pathway has been characterised as the cause of this. Despite improved cytolytic activity and cytokine production, third generation CAR-T cells are more susceptible to AICD as a result of increased FasL expression.

Therefore, avoiding apoptosis triggered via the Fas-FasL pathway offers a significant benefit to adoptively transferred cells in terms of both infiltration (via tumour vasculature) and persistence within the TME.

FasL-Binding Receptor (FLBR)

The present invention relates to a FasL-binding receptor (FLBR) comprising a Fas ectodomain and Tumour Necrosis Factor Receptor (TNFR) endodomain.

The FLBR may have the general structure:

Fas exo-TM-TNFR endo

in which:

Fas exo is the extracellular domain of Fas;

TM is a transmembrane domain; and

TNFR endo is the endodomain of a TNF receptor.

Fas Ectodomain

The sequence of human Fas is available from Uniprot (Accession No. P25445) and shown below as sequence ID No. 49. In this sequence, residues 26-173 form the extracellular domain (SEQ ID No. 50); residues 174-190 form the transmembrane domain (SEQ ID No. 20); and residues 191-335 form the cytoplasmic domain (SEQ ID No. 51). In SEQ ID No. 51, the portion of sequence that is deleted in the truncated Fas described in the Examples (FasΔDD) is underlined.

(human Fas)
SEQ ID No. 49
MLGIWTLLPLVLTSVARLSSKSVNAQVTDINSKGLELRKTVTTVETQNL
EGLHHDGQFCHKPCPPGERKARDCTVNGDEPDCVPCQEGKEYTDKAHFS
SKCRRCRLCDEGHGLEVEINCTRTQNTKCRCKPNFFCNSTVCEHCDPCT
KCEHGIIKECTLTSNTKCKEEGSRSNLGWLCLLLLPIPLIVWVKRKEVQ
KTCRKHRKENQGSHESPTLNPETVAINLSDVDLSKYITTIAGVMTLSQV
KGFVRKNGVNEAKIDEIKNDNVQDTAEQKVQLLRNWHQLHGKKEAYDTL
IKDLKKANLCTLAEKIQTIILKDITSDSENSNFRNEIQSLV
(Fas extracellular domain)
SEQ ID No. 50
QVTDINSKGLELRKTVTTVETQNLEGLHHDGQFCHKPCPPGERKARDCT
VNGDEPDCVPCQEGKEYTDKAHFSSKCRRCRLCDEGHGLEVEINCTRTQ
NTKCRCKPNFFCNSTVCEHCDPCTKCEHGIIKECTLTSNTKCKEEGSRS
N
SEQ ID No. 51(Fas cytoplasmic domain)
KRKEVQKTCRKHRKENQGSHESPTLNPETVAINLSDVDLSKYITTIAGV
MTLSQVKGFVRKNGVNEAKIDEIKNDNVQDTAEQKVQLLRNWHQLHGKK
EAYDTLIKDLKKANLCTLAEKIQTIILKDITDSENSNFRNEIQSLV

The FLBR of the present invention may comprise the Fas extracellular domain shown as SEQ ID No. 50 or variant thereof which is at least 80, 90, 95 or 99% identical to SEQ ID No. 50 provided that the resultant FLBR molecule competes with endogenous Fas for binding to FasL and has no or reduced capacity to bind FADD.

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

FLBR Transmembrane Domain

The FLBR comprises a transmembrane domain that spans the membrane. The transmembrane domain may be any protein structure which is thermodynamically stable in a membrane e.g. does not dimerize. This is typically an alpha helix comprising of several hydrophobic residues. The transmembrane domain of any transmembrane protein can be used to supply a transmembrane portion. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e., a polypeptide sequence predicted to form a hydrophobic alpha helix of sufficient length to span the membrane, an artificially designed TM domain may also be used (U.S. Pat. No. 7,052,906 B1 describes transmembrane components).

The transmembrane domain may comprise a hydrophobic alpha helix. The transmembrane domain may be derived from Fas. The transmembrane domain may comprise the sequence shown as SEQ ID NO: 20 or a variant thereof having at least 80% sequence identity.

(Fas transmembrane domain)
SEQ ID NO: 20
LGWLCLLLLPIPLIVVW

The variant may have at least 90, 95, 98 or 99% sequence identity with SEQ ID NO: 20, provided that the variant sequence retains the capacity to traverse the membrane.

The transmembrane domain may be based on the transmembrane domain from a TNFR, for example a TNFR as described herein. Suitably the transmembrane domain may be based on the same TNFR as the endodomain present in the FLBR.

Suitably, the transmembrane domain may comprise any one of SEQ ID NO: 20-34 or a variant thereof having at least 80% sequence identity. The variant may have at least 90, 95, 98 or 99% sequence identity with SEQ ID NO: 21-34, provided that the variant sequence retains the capacity to traverse the membrane.

(DcR2 transmembrane domain)
SEQ ID NO: 21
YLIIIVVLVIILAWWGFSC
(GITR transmembrane domain)
SEQ ID NO: 22
LGWLTVVLLAVAACVLLLTSA
(CD30 transmembrane domain)
SEQ ID NO: 23
PVLFWWILVLVVVVGSSAFLL
(CD8 transmembrane domain)
SEQ ID NO: 24
APTIASQPLSLRPEACRPAAGGAVHTRGLDFACD
(CD28 transmembrane domain)
SEQ ID NO: 25
FLFVLLGVGSMGVAAIVWGAW
(4-1BB transmembrane domain)
SEQ ID NO: 26
IISFFLALTSTALLFLLFFLTLRFSVV
(DR3 transmembrane domain)
SEQ ID NO: 27
MFWWQVLLAGLVVPLLLGATL
(OX40 transmembrane domain)
SEQ ID NO: 28
VAAILGLGLVLGLLGPLAILL
(CD70 transmembrane domain)
SEQ ID NO: 29
VLRAALVPLVAGLVICLVVCI
(CD40 transmembrane domain)
SEQ ID NO: 30
ALVVIPIIFGILFAILLVLVFI
(XEDAR transmembrane domain)
SEQ ID NO: 31
LVALVSSLLVVF TLAFLGLFF
(Fn14 transmembrane domain)
SEQ ID NO: 32
ILGGALSLTFVLGLLSGFLVW
(BCMA transmembrane domain)
SEQ ID NO: 33
ILWTCLGLSLIISLAVFVLMFLL
(CD27 transmembrane domain)
SEQ ID NO: 34
ILVIFSGMFLVFTLAGALFLH
TNFR ENDODOMAIN

The structural motifs in the cytoplasmic domains of TNF superfamily categorize them into two groups based on their signalling properties: those contain a death domain (DD) and others that engage TNFR-associated factors (TRAFs). There is a third group which lack a membrane-anchor domain and are proteolytically cleaved from the surface or are anchored via glycolipid linkage and are termed “decoy receptors”.

A list of TNFRs is given in Table 1.

TABLE 1

| Protein (member #) | Synonyms | Gene | Ligand(s) |
|---|---|---|---|
| Tumornecrosis | CD120b | TNFRSF1B | TNF-alpha |
| factor receptor 2 | | | (cachectin) |
| Lymphotoxin beta | CD18 | LTBR | Lymphotoxin beta |
| receptor | | | (TNF-C) |
| OX40 | CD134 | TNFRSF4 | OX40L |
| CD40 | Bp50 | CD40 | CD154 |
| Fas receptor | Apo-1, CD95 | FAS | FasL |
| Decoy receptor 3 | TR6, M68 | TNFRSF6B | FasL, LIGHT, TL1A |
| (DcR3) | | | |
| CD27 | S152, Tp55 | CD27 | CD70, Siva |
| CD30 | Ki-1 | TNFRSF8 | CD153 |
| 4-1BB | CD137 | TNFRSF9 | 4-1 BB ligand |
| Death receptor 4 | TRAILR1, Apo-2, | TNFRSF10A | TRAIL |
| | CD261 | | |
| Death receptor 5 | TRAILR2, CD262 | TNFRSF10B | TRAIL |
| Decoy receptor 1 | TRAILR3, LIT, TRID, | TNFRSF10C | TRAIL |
| | CD263 | | |
| Decoy receptor 2 | TRAILR4, TRUNDD, | TNFRSF10D | TRAIL |
| (DcR3) | CD264 | | |
| RANK | CD265 | TNFRSF11A | RANKL |
| Osteoprotegerin | OCIF, TR1 | TNFRSF11B | |
| TWEAK receptor | Fn14, CD266 | TNFRSF12A | TWEAK |
| TACI | IGAD2, CD267 | TNFRSF13B | APRIL, BAFF, |
| | | | CAM LG |
| BAFF receptor | CD268 | TNFRSF13C | BAFF |
| (BAFF-R) | | | |
| Herpesvirus entry | ATAR, TR2, CD270 | TNFRSF14 | LIGHT |
| mediator (HVEM) | | | |
| Nerve growth factor | p75NTR, CD271 | NGFR | NGF, BDNF, NT-3, |
| receptor | | | NT-4 |
| B-cell maturation | TNFRSF13A, | TNFRSF17 | BAFF |
| antigen (BCMA) | CD269 | | |
| Glucocorticoid- | AITR, CD357 | TNFRSF18 | GITR ligand |
| induced TNFR- | | | |
| related (GITR) | | | |
| TROY | TAJ, TRADE | TNFRSF19 | unknown |
| Death receptor 6 | CD358 | TNFRSF21 | |
| Death receptor 3 | Apo-3,TRAMP, | TNFRSF25 | TL1A |
| | LARD, WS-1 | | |
| Ectodysplasin A2 | XEDAR | EDA2R | EDA-A2 |
| receptor | | | |

The FLBR of the present invention may comprise the endodomain of a TNFR or the signalling portion thereof. The TNFR may be selected from the group consisting of GITR, DcR2, CD30, XEDAR, CD40, CD27, BCMA or Fn14.

Glucocorticoid induced TNF-receptor (GITR)

The FLBR may comprise the endodomain of GITR (Uniprot ID: Q9Y5U5). GITR is a cell surface receptor constitutively expressed on T cells, with its surface expression increased upon CD3/28 stimulation. It is a co-stimulatory receptor with its activation leading to NFkB and MAP-kinase signalling, via TRAF2/5 recruitment, resulting in upregulation of CD25 and secretion of IL-2 and IFNγ.

GITR endodomain is shown in SEQ ID NO: 1. The FLBR may comprise SEQ ID NO: 1 or a variant thereof which has at least 80, 85, 90, 95, 98 or 99% sequence identity to SEQ ID NO: 1 and retains the ability to induce GITR-mediated signalling.

(GITR endodomain)
SEQ ID NO: 1
QLGLHIWQLRSQCMWPRETQLLLEVPPSTEDARSCQFPEEERGERSAEE
KGRLGDLWV

CD30 Endodomain

The FLBR may comprise the endodomain of CD30 (Uniprot ID: P28908). CD30, also known as TNFRSF8, is a cell membrane protein of the TNFR family and a tumour marker. The receptor is expressed by activated T and B cells TRAF2 and TRAF5 can interact with this receptor and mediate the signal transduction that leads to the activation of NF-kappa-B. It is a positive regulator of apoptosis, and also has been shown to limit the proliferative potential of autoreactive CD8 effector T cells and protect the body against autoimmunity. Two alternatively spliced transcript variants of this gene encoding distinct isoforms have been reported.

CD30 endodomain is shown in SEQ ID NO: 2. The FLBR may comprise SEQ ID NO: 2 or a variant thereof which has at least 80, 85, 90, 95, 98 or 99% sequence identity to SEQ ID NO: 2 and retains the ability to induce CD30-mediated signalling.

(CD30 endodomain)
SEQ ID NO: 2
HRRACRKRIRQKLHLCYPVQTSQPKLELVDSRPRRSSTQLRSGASVTEP
VAEERGLMSQPLMETCHSVGAAYLESLPLQDASPAGGPSSPRDLPEPRV
STEHTNNKIEKIYIMKADTVIVGTVKAELPEGRGLAGPAEPELEEELEA
DHTPHYPEQETEPPLGSCSDVMLSVEEEGKEDPLPTAASGK

XEDAR Endodomain

The FLBR may comprise the endodomain of XEDAR (Uniprot ID: QPHAV5). XEDAR, also known as TNFRSF27 or EDA2R (Ectodysplasin A2 receptor) is a type III transmembrane protein of the TNFR (tumour necrosis factor receptor) superfamily and contains 3 cysteine-rich repeats and a single transmembrane domain but lacks an N-terminal signal peptide. This protein mediates the activation of the NF-kappa-B and JNK pathways. Activation is to be mediated by binding to TRAF3 and TRAF6.

XEDAR endodomain is shown in SEQ ID NO: 3. The FLBR may comprise SEQ ID NO: 3 or a variant thereof which has at least 80, 85, 90, 95, 98 or 99% sequence identity to SEQ ID NO: 3 and retains the ability to induce XEDAR-mediated signalling.

(XEDAR endodomain)
SEQ ID NO: 3
TSNTKCKEEGSRSNLGWLCLLLLPIPLIVWVLYCKQFFNRHCQRGGLLQ
FEADKTAKEESLFPVPPSKETSAESQVSENIFQTQPLNPILEDDCSSTS
GFPTQESFTMASCTSESHSHWVHSPIECTELDLQKFSSSASYTGAETLG
GNTVESTGDRLELNVPFEVPSP

CD40 Endodomain

The FLBR may comprise the endodomain of CD40 (Uniprot ID: P25942). CD40 (Cluster of differentiation 40) or TNFRSF5 (tumour necrosis factor superfamily member 5) is a costimulatory protein found on antigen-presenting cells and is required for their activation. This receptor has been found to be essential in mediating a broad variety of immune and inflammatory responses including T cell-dependent immunoglobulin class switching, memory B cell development, and germinal center formation. CD40 transduces TRAF6- and MAP3K8-mediated signals that activate ERK in macrophages and B cells, leading to induction of immunoglobulin secretion.

CD40 endodomain is shown in SEQ ID NO: 4. The FLBR may comprise SEQ ID NO: 4 or a variant thereof which has at least 80, 85, 90, 95, 98 or 99% sequence identity to SEQ ID NO: 4 and retains the ability to induce CD40-mediated signalling.

(CD40 endodomain)
SEQ ID NO: 4
KKVAKKPTNKAPHPKQEPQEINFPDDLPGSNTAAPVQETLHGCQPVTQE
DGKESRISVQERQ

CD27 Endodomain

The FLBR may comprise the endodomain of CD27 (Uniprot ID: QPHAV5). CD27, also known as TNFRSF7 (tumour necrosis factor receptor superfamily 7) or T-cell activation antigen CD27, is a transmembrane protein required for generation and long-term maintenance of T cell immunity. It binds to ligand CD70, and plays a key role in regulating B-cell activation and immunoglobulin synthesis. This receptor transduces signals that lead to the activation of NF-kappaB and MAPK8/JNK. Adaptor proteins TRAF2 and TRAF5 have been shown to mediate the signalling process of this receptor. CD27-binding protein (SIVA), a proapoptotic protein, can bind to this receptor and is thought to play an important role in the apoptosis induced by this receptor.

CD27 endodomain is shown in SEQ ID NO: 5. The FLBR may comprise SEQ ID NO: 5 or a variant thereof which has at least 80, 85, 90, 95, 98 or 99% sequence identity to SEQ ID NO: 5 and retains the ability to induce GITR-mediated signalling.

(CD27 endodomain)
SEQ ID NO: 5
QRRKYRSNKGESPVEPAEPCHYSCPREEEGSTIPIQEDYRKPEPACSP

BCMA Endodomain

The FLBR may comprise the endodomain from BCMA (Uniprot ID: QO2223). BCMA (B-cell maturation antigen), also known as TNFRSF17 (tumour necrosis factor receptor superfamily member 17) is a protein that in humans is encoded by the TNFRSF17 gene. TNFRSF17 is a cell surface receptor of the TNF receptor superfamily which recognizes B-cell activating factor (BAFF/TNFSF13B) and A proliferation inducing ligand (APRIL/TNFSF13). This receptor promotes B-cell survival and plays a role in the regulation of humoral immunity. It also activates NF-kappa-B and JNK.

BCMA endodomain is shown in SEQ ID NO: 6. The FLBR may comprise SEQ ID NO: 6 or a variant thereof which has at least 80, 85, 90, 95, 98 or 99% sequence identity to SEQ ID NO: 6 and retains the ability to induce GITR-mediated signalling.

(BCMA endodomain)
SEQ ID NO: 6
RKINSEPLKDEFKNTGSGLLGMANIDLEKSRTGDEIILPRGLEYTVEEC
TCEDCIKSKPKVDSDHCFPLPAMEEGATILVTTKTNDYCKSLPAALSAT
EIEKSISAR

Fn14 Endodomain

The FLBR may comprise the endodomain of Fn14 (Uniprot ID: Q9NP84. Fn14, also known as TNFRSF12A is a receptor for TNFSF12/TWEAK and a weak inducer of apoptosis in some cell types. It also promotes angiogenesis and the proliferation of endothelial cells and may modulate cellular adhesion to matrix proteins.

Fn14 endodomain is shown in SEQ ID NO: 7. The FLBR may comprise SEQ ID NO: 7 or a variant thereof which has at least 80, 85, 90, 95, 98 or 99% sequence identity to SEQ ID NO: 7 and retains the ability to induce Fn14-mediated signalling.

(Fn14 endodomain)
SEQ ID NO: 7
RRCRRREKFTTPIEETGGEGCPAVALIQ

DcR2 (Decoy Receptor 2)

The FLBR may comprise part of the endodomain of DcR2 (Uniprot ID: Q9UBN6). DcR2, also known as TRAIL4 or TNFRSF10D (tumour necrosis factor receptor superfamily 10D) is a cell surface receptor for the cytotoxic ligand TRAIL.

The FLBR of the invention may comprise an intracellular domain of DcR2 which lacks functional death domain. For example, the DcR2 endodomain may comprise a truncated death domain, which is unable to bind FADD. Fusing the extracellular binding domain of Fas with the intracellular domain of DcR2 having a non-functional death domain results in an FLBR which competes with Fas for binding to FasL but is unable to bind to FADD, inhibiting the FasL-induced apoptosis pathway.

The DcR2 intracellular domain induces NF-κB signalling, providing pro-survival and anti-apoptotic functions, which is a feature exploited by cancers. Therefore, combining the Fas ectodomain to the DcR2 endodomain in the FLBR of the invention is beneficial in that it negates pro-apoptotic signals induced by FasL and converts a pro-apoptotic signal into a pro-survival outcome via NFκB.

DcR2 endodomain with a truncated death domain is shown in SEQ ID NO: 8. The FLBR may comprise SEQ ID NO: 8 or a variant thereof which has at least 80, 85, 90, 95, 98 or 99% sequence identity to SEQ ID NO: 8 and retains the ability to induce DcR2-mediated signalling.

(DcR2 endodomain)
SEQ ID NO: 8
RKKFISYLKGICSGGGGGPERVHRVLFRRRSCPSRVPGAEDNARNETLS
NRYLQPTQVSEQEIQGQELAELTGVTVESPEEPQRLLEQAEAEGCQRRR
LLVPVNDADSADISTLLDASATLEEGHAKETIQDQLVGSEKLFYEEDEA
GSATSCL

The FLBR of the present invention may comprise more than one TNFR endodomain. For example, the Fas ectodomain and TNFR endodomain of the FLBR may be fused to the signalling portion of an additional TNFR endodomain selected from any one of the TNFR endodomains listed in Table 1. The FLBR construct may comprise, for example, Fas receptor ectodomain and two TNFR endodomains (e.g. Fas-CD30-41 BB or Fas-CD30-OX40).

Below is a list of complete FLBR sequences including a signal peptide (in normal text); the Fas ectodomain (in bold); a transmembrane domain (in italics) and a TNFR endodomain (underlined). The FLBR may comprise one of these full sequences or a portion thereof: for example, it may comprise the Fas ectodomain and TNFR combination but with a different signal peptide and/or transmembrane domain.

(Human Fas-Dcr2)
SEQ ID NO: 35
MGWSCIILFLVATATGVHSQVTDINSKGLELRKTVTTVETQNLEGLHHD
GQFCHKPCPPGERKARDCTVNGDEPDCVPCQEGKEYTDKAHFSSKCRRC
RLCDEGHGLEVEINCTRTQNTKCRCKPNFFCNSTVCEHCDPCTKCEHGI
IKECTLTSNTKCKEEGSRSNLGWLCLLLLPIPLIVWVRKKFISYLKGIC
SGGGGGPERVHRVLFRRRSCPSRVPGAEDNARNETLSNRYLQPTQVSEQ
EIQGQELAELTGVTVESPEEPQRLLEQAEAEGCQRRRLLVPVNDADSAD
ISTLLDASATLEEGHAKETIQDQLVGSEKLFYEEDEAGSATSCL
(Human Fas-GITR)
SEQ ID NO: 36
MGWSCIILFLVATATGVHSQVTDINSKGLELRKTVTTVETQNLEGLHHD
GQFCHKPCPPGERKARDCTVNGDEPDCVPCQEGKEYTDKAHFSSKCRRC
RLCDEGHGLEVEINCTRTQNTKCRCKPNFFCNSTVCEHCDPCTKCEHGI
IKECTLTSNTKCKEEGSRSNLGWLCLLLLPIPLIVWVRKQLGLHIWQLR
SQCMWPRETQLLLEVPPSTEDARSCQFPEEERGERSAEEKGRLGDLWV
(Human Fas-CD30)
SEQ ID NO: 37
MGWSCIILFLVATATGVHSQVTDINSKGLELRKTVTTVETQNLEGLHHD
GQFCHKPCPPGERKARDCTVNGDEPDCVPCQEGKEYTDKAHFSSKCRRC
RLCDEGHGLEVEINCTRTQNTKCRCKPNFFCNSTVCEHCDPCTKCEHGI
IKECTLTSNTKCKEEGSRSNLGWLCLLLLPIPLIVWVRKHRRACRKRIR
QKLHLCYPVQTSQPKLELVDSRPRRSSTQLRSGASVTEPVAEERGLMSQ
PLMETCHSVGAAYLESLPLQDASPAGGPSSPRDLPEPRVSTEHTNNKIE
KIYIMKADTVIVGTVKAELPEGRGLAGPAEPELEEELEADHTPHYPEQE
TEPPLGSCSDVMLSVEEEGKEDPLPTAASGK
(Human Fas-XEDAR)
SEQ ID NO: 38
MGWSCIILFLVATATGVHSQVTDINSKGLELRKTVTTVETQNLEGLHHD
GQFCHKPCPPGERKARDCTVNGDEPDCVPCQEGKEYTDKAHFSSKCRRC
RLCDEGHGLEVEINCTRTQNTKCRCKPNFFCNSTVCEHCDPCTKCEHGI
IKECTLTSNTKCKEEGSRSNLGWLCLLLLPIPLIVWVLYCKQFFNRHCQ
RGGLLQFEADKTAKEESLFPVPPSKETSAESQVSENIFQTQPLNPILED
DCSSTSGFPTQESFTMASCTSESHSHWVHSPIECTELDLQKFSSSASYT
GAETLGGNTVESTGDRLELNVPFEVPSP
(Human Fas-CD27)
SEQ ID NO: 39
MGWSCIILFLVATATGVHSQVTDINSKGLELRKTVTTVETQNLEGLHHD
GQFCHKPCPPGERKARDCTVNGDEPDCVPCQEGKEYTDKAHFSSKCRRC
RLCDEGHGLEVEINCTRTQNTKCRCKPNFFCNSTVCEHCDPCTKCEHGI
IKECTLTSNTKCKEEGSRSNLGWLCLLLLPIPLIVWVQRRKYRSNKGES
PVEPAEPCHYSCPREEEGSTIPIQEDYRKPEPACSP
(Human Fas-BCMA)
SEQ ID NO: 40
MGWSCIILFLVATATGVHSQVTDINSKGLELRKTVTTVETQNLEGLHHD
GQFCHKPCPPGERKARDCTVNGDEPDCVPCQEGKEYTDKAHFSSKCRRC
RLCDEGHGLEVEINCTRTQNTKCRCKPNFFCNSTVCEHCDPCTKCEHGI
IKECTLTSNTKCKEEGSRSNLGWLCLLLLPIPLIVWVRKINSEPLKDEF
KNTGSGLLGMANIDLEKSRTGDEIILPRGLEYTVEECTCEDCIKSKPKV
DSDHCFPLPAMEEGATILVTTKTNDYCKSLPAALSATEIEKSISAR
(Human Fas-CD40)
SEQ ID NO: 41
MGWSCIILFLVATATGVHSQVTDINSKGLELRKTVTTVETQNLEGLHHD
GQFCHKPCPPGERKARDCTVNGDEPDCVPCQEGKEYTDKAHFSSKCRRC
RLCDEGHGLEVEINCTRTQNTKCRCKPNFFCNSTVCEHCDPCTKCEHGI
IKECTLTSNTKCKEEGSRSNLGWLCLLLLPIPLIVWVKKVAKKPTNKAP
HPKQEPQEINFPDDLPGSNTAAPVQETLHGCQPVTQEDGKESRISVQER
Q
(Human Fas-Fn14)
SEQ ID NO: 42
MGWSCIILFLVATATGVHSQVTDINSKGLELRKTVTTVETQNLEGLHHD
GQFCHKPCPPGERKARDCTVNGDEPDCVPCQEGKEYTDKAHFSSKCRRC
RLCDEGHGLEVEINCTRTQNTKCRCKPNFFCNSTVCEHCDPCTKCEHGI
IKECTLTSNTKCKEEGSRSNLGWLCLLLLPIPLIVWVRRCRRREKFTTP
IEETGGEGCPAVALIQ

The FLBR of the invention may comprise any one of SEQ ID NO: 35 to 42 or a variant thereof having at least 80% sequence identity. The variant may have at least 80, 85, 90, 95, 98 or 99% sequence identity with SEQ ID NO: 35 to 42, provided that the variant sequence retains the capacity to inhibit FasL-induced apoptosis when expressed by a cell.

Membrane-Bound DcR3

DcR3 (Decoy Receptor 3, TNFRSF6B, Uniprot ID: O95407) is a type I transmembrane glycoprotein which has undergone alternative splicing, resulting in a soluble member of the TNFR super family. This soluble receptor binds to and neutralises the biological functions of FasL, thus inhibiting FasL-induced apoptosis. DcR3 also binds to the ligands TL1A and LIGHT, which are also associated with inducing apoptosis via receptors DR3 and HVEM respectively.

To overcome FasL-induced CAR-T cell apoptosis in the TME, the FLBR of the present invention may comprise the FasL-binding portion of DcR3 which will compete with Fas by binding to FasL. However, since DcR3 is a soluble protein, secretion of DcR3 by CAR-Ts could have confounding effects upon host T cells (neutralising FasL-expressing-host T cell response).

To reduce cell-cell contact of DcR3 with FasL present on the host T cell, DcR3 of the present invention is a membrane bound DcR3. Membrane bound DcR3 may have the general structure:

DcR3-spacer-TM-endo
in which:
DcR3 comprises the FasL binding domain of DcR3
spacer is a spacer sequence connecting DcR3 to the transmembrane domain
TM is a transmembrane domain
endo is an optional intracellular sequence.

The FLBR may comprise a Dcr3 domain having SEQ ID NO: 9 or a variant thereof which have at least 80, 85, 90, 95, 98 or 99% sequence identity to SEQ ID NO: 9 which retains the capacity to bind FasL.

(DcR3)
SEQ ID NO: 9
VAETPTYPWRDAETGERLVCAQCPPGTFVQRPCRRDSPTTCGPCPPRHY
TQFWNYLERCRYCNVLCGEREEEARACHATHNRACRCRTGFFAHAGFCL
EHASCPPGAGVIAPGTPSQNTQCQPCPPGTFSASSSSSEQCQPHRNCTA
LGLALNVPGSSSHDTLCTSCTGFPLSTRVPGAEECERAVIDFVAFQDIS
IKRLQRLLQALEAPEGWGPTPRAGRAALQLKLRRRLTELLGAQDGALLV
RLLQALRVARMPGLERSVRERFLPVH

The structure of DcR3 comprises four N-terminal cysteine-rich domains (CRD) that bind FasL, and a C-terminal heparan-binding domain (HBD) that binds to heparan sulphate proteoglycans (HSPG) (FIG. 2e). It has been shown that the HBD of DcR3, but not the FasL-binding CRDs, induce apoptosis in dendritic cells via cross-linking of HSPGs (You et al., 2008, Blood 111, 1480-1488).

The HBD of DcR3 binds to HSPGs via a binding motif comprising three basic amino acids (K²⁵⁶, R²⁵⁸ and R²⁵⁹). The FLBR of the invention may comprise a mutant version of DcR3 comprising a mutation in one of more or residues K²⁵⁶, R²⁵⁸ and R²⁵⁹. The mutation(s) may reduce or abolish binding to HSPGs. The or each mutation may be a substitution mutation. The or each mutation may involve substituting the amino acid with alanine. The FLBR may comprise the sequence shown as SEQ ID No. 10, which comprises alanine substitutions at each of positions K²⁵⁶, R²⁵⁸ and R²⁵⁹, as shown in bold.

(mutant DcR3)
SEQ ID NO: 10
VAETPTYPWRDAETGERLVCAQCPPGTFVQRPCRRDSPTTCGPCPPRHY
TQFWNLERCRYCNVLCGEREEEARACHATHNRACRCRTGFFAHAGFCLE
HASCPPGAGVIAPGTPSQNTQCQPCPPGTFSASSSSSEQCQPHRNCTAL
GLALNVPGSSSHDTLCTSCTGFPLSTRVPGAEECERAVIDFVAFQDISI
KRLQRLLQALEAPEGWGPTPRAGRAALQLALAARLTELLGAQDGALLVR
LLQALRVARMPGLERSVRERFLPVH

Spacer

The spacer sequence may be any sequence which physically distances the DcR3 domain from the cell membrane and/or provides a degree of flexibility. Spacer sequences commonly used in CARs may be used in the membrane-bound DcR3 FLBR of the present invention. A list of suitable sequences for illustrative purposes only is provided in Table 2.

TABLE 2
Suitable spacer sequences for membrane-bound DcR3

| SEQ IDs | Amino acid sequence |
|---|---|
| SEQ ID NO: | PTTTPAPRPPTPAPTIASQPLSLRPEACRPAAGGAVHT |
| 11 (CD8stk) | RGLDFACD |
| SEQ ID NO: | EPKSCDKTHTCPPCP |
| 12 (Hinge) | |
| SEQ ID NO: | AGSDLGPQMLRELQETNAALQDVRELLRQQVREITFLK |
| 13 (COMP) | NTVMECDACGSGK |
| SEQ ID NO: | KGPSVFPLAPSSKSTSGGTAALGCLVKDYFPEPVTVSW |
| 14 (CH1- | NSGALTSGVHTFPAVLQSSGLYSLSSVVTVPSSSLGTQ |
| Hinge) | TYICNVNHKPSNTKVDKRVEPKSCDKTHTCPPCP |
| SEQ ID NO: | TKGPSVFPLAPSSKSTSGGTAALGCLVKDYFPEPVTVS |
| 15 (CH1- | WNSGALTSGVHTFPAVLQSSGLYSLSSVVTVPSSSLGT |
| Hinge-CH2- | QTYICNVNHKPSNTKVDKRVEPKSCDKTHTCPPCPAPP |
| CH3) | VAGPSVFLFPPKPKDTLMIARTPEVTCWVDVSHEDPEV |
| | KFNWYVDGVEVHNAKTKPREEQYNSTYRVVSVLTVLHQ |
| | DWLNGKEYKCKVSNKALPAPIEKTISKAKGQPREPQVY |
| | TLPPSRDELTKNQVSLTCLVKGFYPSDIAVEWESNGQP |
| | ENNYKTTPPVLDSDGSFFLYSKLTVDKSRWQQGNVFSC |
| | SVMHEALHNHYTQKSLSLSPGK |
| SEQ ID NO: | RTVAAPSVFIFPPSDEQLKSGTASVVCLLNNFYPREAK |
| 16 (HuIG | VQWKVDNALQSGNSQESVTEQDSKDSTYSLSSTLTLSK |
| Kappa) | ADYEKHKVYACEVTHQGLSSPVTKSFNRGEC |
| SEQ ID NO: | AKIEVMYPPPYLDNEKSNGTIIHVKGKHLCPSPLFPGP |
| 17 (CD28 | SKP |
| Stalk) | |

Membrane-bound DcR3 may comprise any one of the spacers shown as SEQ ID NOs: 11 to 17 or a variant thereof which has at least 80, 85, 90, 95, 98 or 99% sequence identity to SEQ ID NOs: 11 to 17.

The FLBR may comprise a short stretch of amino acids linking DcR3 to the spacer. The linker may, for example, comprise between 2 and 10 or between 3 and 5 amino acids. The linker may be the “standard” SDP linker sequence.

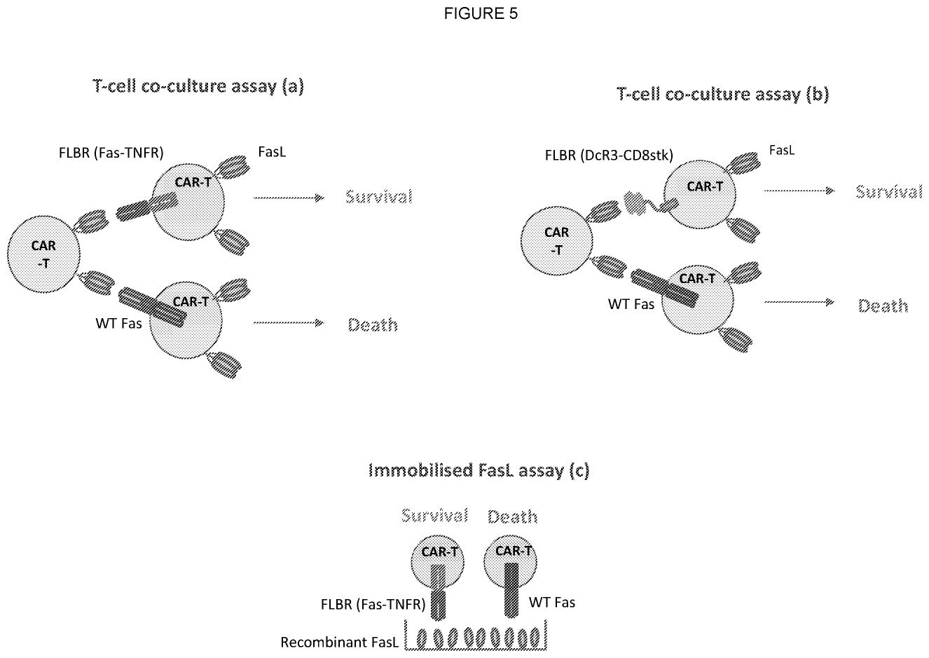

The FLBR described herein may comprise a modified DcR3 that is bound to the plasma membrane via a CD8 transmembrane stalk (CD8stk) as depicted in FIG. 2d and FIG. 5b.

Membrane-Bound DcR3 TM Domain

The membrane-bound DcR3 also comprises a transmembrane domain. As explained above, the transmembrane domain may be any protein structure which is thermodynamically stable in a membrane and is typically an alpha helix comprising of several hydrophobic residues. The transmembrane domain may be derived from any transmembrane protein. Where the spacer is derived from a transmembrane protein, such as CD8 or CD28, the TM domain may be conveniently derived from the same protein.

The sequences of CD28 and CD8a are shown below as SEQ ID No. 52 and 53 respectively.

(CD28 TM domain)
SEQ ID No. 52
FWVLVVVGGVLACYSLLVTVAFIIFWV
(CD8 TM domain)
SEQ ID No. 53
IYIWAPLAGTCGVLLLSLVIT

The transmembrane domain may comprise the sequence shown as SEQ ID NO: 52 or 53 or a variant thereof having at least 90, 95 or 99% sequence identity which retains the capacity to span the membrane.

Membrane Bound DcR3 Endodomain

An FLBR comprising membrane-bound DcR3 variants may include an intracellular polar anchor which provides a polar region in close proximity to the plasma membrane. A polar anchor sequence is shown as SEQ ID No. 18 below.

Alternatively or in addition, the FLBR may include an intracellular rigid linker. This sequence is less flexible that the commonly used Glycine-Serine linker and is advantageous at the C-terminal end of a recombinant protein sequence. A rigid linker sequence is shown as SEQ ID No. 19 below.

(Polar anchor)
SEQ ID NO: 18
RKKR
(Rigid linker with truncations)
SEQ ID NO: 19
LEAEAAAKEAAAKEAAAKEAAAKALEAEAAAKEAAAKEAAAKEAAAKALE

Membrane-bound DcR3 feature of the FLBR of the present invention may also comprise a TNFR endodomain in addition to or instead of the polar anchor/rigid linker. The TNFR endodomain may be selected from any one of the TNFR listed in Table 1. The TNFR endodomain may comprise one of the sequences shown as SEQ ID No. 1 to 8.

Below are two complete FLBR sequences based on membrane-bound DcR3. The sequences include a signal peptide (in normal text); DcR3 (in bold); a standard linker (in bold and underlined); a spacer (in italics); a transmembrane domain (underlined); a polar anchor (double underlined); and a rigid linker (in bold and italics). The FLBR may comprise one of these full sequences or a portion thereof: for example, it may comprise DcR3 and polar anchor/rigid linker but with a different signal peptide, spacer and/or transmembrane domain.

(Human DcR3-CD8 stalk/TM/rigid linker)
SEQ ID NO: 43
METDTLILWVLLLLVPGSTGVAETPTYPWRDAETGERLVCAQCPPGTF
VQRPCRRDSPTTCGPCPPRHYTQFWNYLERCRYCNVLCGEREEEARAC
HATHNRACRCRTGFFAHAGFCLEHASCPPGAGVIAPGTPSQNTQCQPC
PPGTFSASSSSSEQCQPHRNCTALGLALNVPGSSSHDTLCTSCTGFPL
STRVPGAEECERAVIDFVAFQDISIKRLQRLLQALEAPEGWGPTPRAG
RAALQQKLRRRLTELLGAQDGALLVRLLQALRVARMPGLERSVRERFL
PVH [symbol] TTTPAPRPPTPAPTIASQPLSLRPEACRPAAGGAVHTRGLD
FACDIYIWAPLAGTCGVLLLSLVITLYCRKKRS

[symbol]

[symbol]

[symbol]

(Human mutant DcR3-CD8 stalk/TM/riqid linker)
SEQ ID NO: 44
METDTLILWVLLLLVPGSTGVAETPTYPWRDAETGERLVCAQCPPGTF
VQRPCRRDSPTTCGPCPPRHYTQFWNYLERCRYCNVLCGEREEEARAC
HATHNRACRCRTGFFAHAGFCLEHASCPPGAGVIAPGTPSQNTQCQPC
PPGTFSASSSSSEQCQPHRNCTALGLALNVPGSSSHDTLCTSCTGFPL
STRVPGAEECERAVIDFVAFQDISIKRLQRLLQALEAPEGWGPTPRAG
RAALQLALAARLTELLGAQDGALLVRLLQALRVARMPGLERSVRERFL
PVH [symbol] TTTPAPRPPTPAPTIASQPLSLRPEACRPAAGGAVHTRGLD
FACDIYIWAPLAGTCGVLLLSLVITLYCRKKRS

[symbol]

[symbol]

The FLBR of the invention may comprise SEQ ID NO: 43 or 44 or a variant thereof having at least 80% sequence identity. The variant may have at least 80, 85, 90, 95, 98 or 99% sequence identity with SEQ ID NO: 43 or 44, provided that the variant sequence retains the capacity to inhibit FasL-induced apoptosis when expressed by a cell.

Nucleic Acid Sequence

The present invention provides a nucleic acid sequence which encodes a FasL-binding receptor (FLBR) comprising;

(i) a Fas ectodomain and a TNFR endodomain, wherein the TNFR endodomain comprises the signalling portion of the decoy receptor 2 (DcR2), GITR, CD30, XEDAR, CD40, CD27, BCMA or Fn14 endodomain, or

(ii) a membrane-bound decoy receptor 3 (DcR3).

The nucleic acid sequence is an “exogenous polynucleotide” in the sense that the polynucleotide which expresses the TNFR is not part of the endogenous genome of the cell. For example, the exogenous polynucleotide may be part of an engineered nucleic acid construct or a vector.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Nucleic Acid Construct

In one aspect the present invention provides a nucleic acid construct which comprises: (i) a first nucleic acid sequence which encodes i) a chimeric antigen receptor (CAR) or a transgenic T-cell receptor (TCR); and (ii) a second nucleic acid sequence as defined above

The nucleic acid construct may have the general structure:

CAR/TCR-coexpr-FLBR; or

FLBR-coexpr-CAR/TCR

in which:

CAR/TCR is a nucleic acid sequence encoding a CAR or TCR;

coexpr is a nucleic acid sequence enabling co-expression of the CAR/TCR and FLBR as separate polypeptides; and

FLBR is a nucleic acid sequence encoding a FasL-binding receptor as described herein.

In the structure above, “coexpr” is a nucleic acid sequence enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No. 54 (RAEGRGSLLTCGDVEENPGP).

The present invention further provides a kit comprising: (i) a first nucleic acid sequence which encodes i) a chimeric antigen receptor (CAR) or a transgenic T-cell receptor (TCR); and (ii) a second nucleic acid sequence which is an exogenous polynucleotide capable of expressing a FasL-binding receptor (FLBR) as defined herein.

Chimeric Antigen Receptor (CAR)

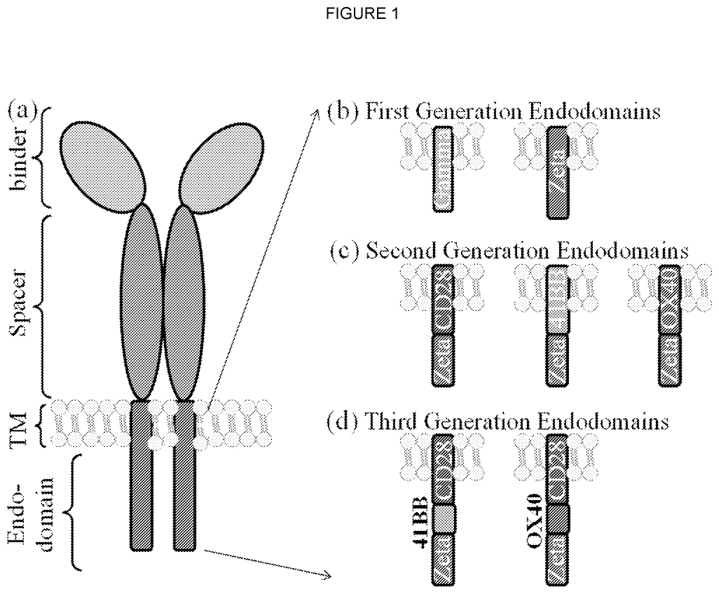

Classical CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first-generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 4-1 BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus, the CAR directs the specificity and cytotoxicity of the T cell towards cells expressing the targeted antigen.

Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, some of which are shown in the following Table 3. The antigen-binding domain used in the present invention may be a domain which is capable of binding a TAA as indicated therein.

TABLE 3

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Transmembrane Domain

The transmembrane domain is the sequence of a classical CAR that spans the membrane. It may comprise a hydrophobic alpha helix. The transmembrane domain may be derived from any transmembrane protein or may be synthetic, as explained above. The TM domain of the CAR may be derived from CD28, which gives good receptor stability. Alternatively the transmembrane domain may be derived from the melanosomal protein Tryp-1.

Signal Peptide

The CAR may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs. The CAR may comprise one of the spacers listed in Table 2.

Intracellular Signalling Domain

The intracellular signalling domain is the signal-transmission portion of a classical CAR. The intracellular signalling domain may be or comprise a T cell signalling domain.

The intracellular signalling domain may comprise one or more immunoreceptor tyrosine-based activation motifs (ITAMs). An ITAM is a conserved sequence of four amino acids that is repeated twice in the cytoplasmic tails of certain cell surface proteins of the immune system.

The motif contains a tyrosine separated from a leucine or isoleucine by any two other amino acids, giving the signature YxxL/I. Two of these signatures are typically separated by between 6 and 8 amino acids in the tail of the molecule (YxxL/Ix₍₆₋₈₎YxxL/I).

ITAMs are important for signal transduction in immune cells. Hence, they are found in the tails of important cell signalling molecules such as the CD3 and ζ-chains of the T cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signalling pathways of the cell.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. There are two main types of co-stimulatory signals: those that belong the Ig family (CD28, ICOS) and the TNF family (OX40, 41BB, CD27, GITR etc—see Table 1). For example, chimeric CD28 and OX40 can be used with CD3-Zeta to transmit a proliferative/survival signal, or all three can be used together (illustrated in FIG. 1B).

The endodomain may comprise the sequence shown as SEQ ID NO: 45 to 48 or a variant thereof having at least 80, 85, 90, 95, 98 or 99% sequence identity, provided that the variant sequence retains the capacity to transmit an activating signal to the cell.

(CD3-ζ endodomain)
SEQ ID NO: 45
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR
(4-1BB and CD3-ζ endodomains)
SEQ ID NO: 46
MGNSCYNIVATLLLVLNFERTRSLQDPCSNCPAGTFCDNNRNQICSPC
PPNSFSSAGGQRTCDICRQCKGVFRTRKECSSTSNAECDCTPGFHCLG
AGCSMCEQDCKQGQELTKKGCKDCCFGTFNDQKRGICRPWTNCSLDGK
SVLVNGTKERDVVCGPSPADLSPGASSVTPPAPAREPGHSPQIISFFL
ALTSTALLFLLFFLTLRFSVVKRGRKKLLYIFKQPFMRPVQTTQEEDG
CSCRFPEEEEGGCELRVKFSRSADAPAYQQGQNQLYNELNLGRREEYD
VLDKRRGRDPEMGGKPQRRKNPQEGLYNELQKDKMAEAYSEIGMKGER
RRGKGHDGLYQGLSTATKDTYDALHMQALPPR
(CD28 and CD3-ζ endodomains)
SEQ ID NO: 47
SKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQE
GLYNELQKDKMAEAYSEIGMKGERRRGKGHDGLYQGLSTATKDTYDAL
HMQALPPR
(CD28, OX40 and CD3-ζ endodomains)
SEQ ID NO: 48
SKRSRLLHSDYMNMTPRRPGPTRKHYQPYAPPRDFAAYRSRDQRLPPD
AHKPPGGGSFRTPIQEEQADAHSTLAKIRVKFSRSADAPAYQQGQNQL
YNELNLGRREEYDVLDKRRGRDPEMGGKPRRKNPQEGLYNELQKDKMA
EAYSEIGMKGERRRGKGHDGLYQGLSTATKDTYDALHMQALPPR

Transgenic T-Cell Receptor (TCR)

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example the genes for engineered TCRs may be reintroduced into autologous T cells and transferred back into patients for T cell adoptive therapies. Such ‘heterologous’ TCRs may also be referred to herein as ‘transgenic TCRs’.

The present invention relates to a cell which co-expresses a transgenic TCR and a FLBR as defined herein.

Vector

The present invention also provides a vector, or kit of vectors, which comprises one or more nucleic acid sequence(s) encoding a FLBR according to the invention. Such a vector may be used to introduce the nucleic acid sequence(s) into a host cell so that it expresses a CAR/TCR and an FLBR as defined herein.

The vector may, for example, be a plasmid or a viral vector, such as a retroviral vector or a lentiviral vector, or a transposon based vector or synthetic mRNA.

The vector may be capable of transfecting or transducing a cell, such as a T cell or a NK cell

Cell

The present invention provides a cell which co-expresses a CAR/TCR and FLBR as defined herein.

The cell may comprise a nucleic acid or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Cells according to the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

In all these embodiments, chimeric polypeptide-expressing cells are generated by introducing DNA or RNA coding for the chimeric polypeptide by one of many means including transduction with a viral vector, transfection with DNA or RNA.

The cell of the invention may be an ex vivo cell from a subject. The cell may be from a peripheral blood mononuclear cell (PBMC) sample. The cells may be activated and/or expanded prior to being transduced with nucleic acid encoding the molecules providing the chimeric polypeptide according to the first aspect of the invention, for example by treatment with an anti-CD3 monoclonal antibody.

The cell of the invention may be made by:
(i) isolation of a cell-containing sample from a subject or other sources listed above; and
(ii) transduction or transfection of the cells with one or more a nucleic acid sequence(s) encoding CAR/TCR and an FLBR as defined herein.

The cells may then by purified, for example, selected on the basis of expression of the antigen-binding domain of the CAR/TCR or on the expression of Fas ectodomain.

Pharmaceutical Composition

The present invention also relates to a pharmaceutical composition containing a cell or plurality of cells of the present invention. In particular, the invention relates to a pharmaceutical composition containing a cell according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering a cell of the present invention (for example in a pharmaceutical composition as described above) to a subject.

Suitably, the present methods for treating and/or preventing a disease may comprise administering a cell of the invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:

(i) isolating a cell-containing sample;

(ii) transducing or transfecting such cells with a nucleic acid sequence or vector provided by the present invention;

(iii) administering the cells from (ii) to a subject.

The present invention provides a cell of the present invention for use in treating and/or preventing a disease

The invention also relates to the use of a cell in the manufacture of a medicament for the treatment and/or prevention of a disease

The disease to be treated and/or prevented by the method of the present invention may be cancer.

The cancer may be such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease may be Multiple Myeloma (MM), B-cell Acute Lymphoblastic Leukaemia (B-ALL), Chronic Lymphocytic Leukaemia (CLL), Neuroblastoma, T-cell acute Lymphoblastic Leukaema (T-ALL) or diffuse large B-cell lymphoma (DLBCL).

The cell, in particular the CAR cell, of the present invention may be capable of killing target cells, such as cancer cells. The target cell may be recognisable by expression of a TAA, for example the expression of a TAA provided above in Table 3. The cancer may be a cancer listed in Table 3.

Method of Making a Cell

CAR or transgenic TCR-expressing cells of the present invention may be generated by introducing DNA or RNA coding for the CAR or TCR and a FasL-binding receptor (FLBR) by one of many means including transduction with a viral vector, transfection with DNA or RNA.

The cell of the invention may be made by:

(i) isolation of a cell-containing sample from a subject or one of the other sources listed above; and

(ii) transduction or transfection of the cells with one or more a nucleic acid sequence(s) or nucleic acid construct as defined above in vitro or ex vivo.

The cells may then by purified, for example, selected on the basis of expression of the antigen-binding domain of the antigen-binding polypeptide.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—In Vitro Testing of Constructs which Bind to FasL

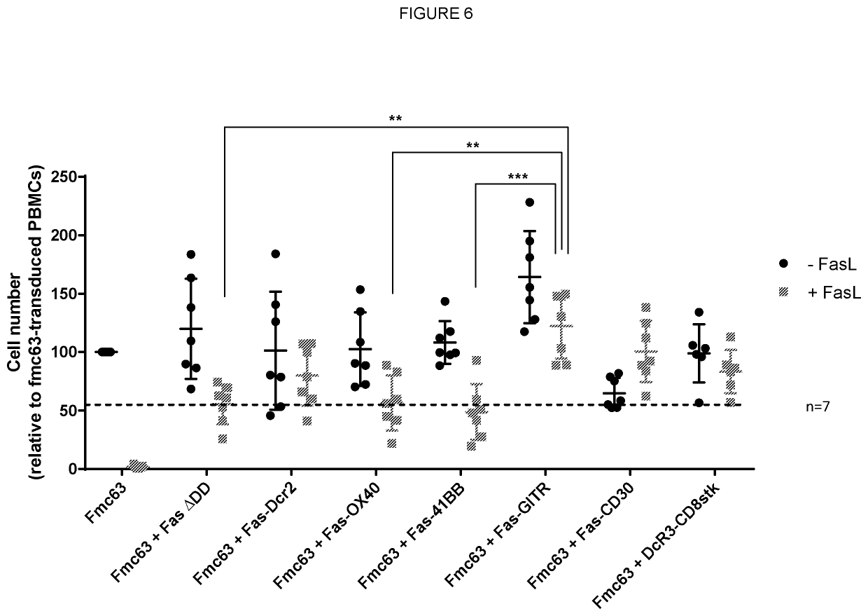

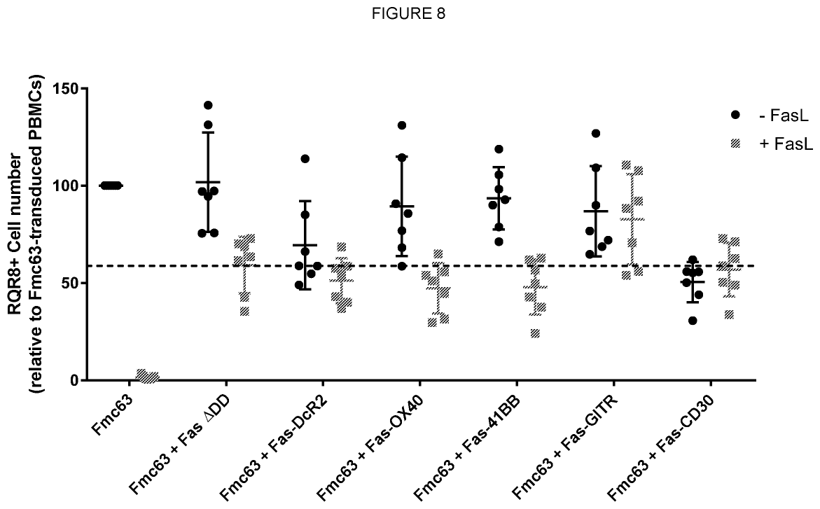

A panel of cells was created expressing the constructs listed in Table 4. All cells expressed a second generation anti-CD19 CAR (Fmc63). Two versions of the panel of cells were created: one in which the construct included a gene encoding Fas ligand, so the cells co-expressed FasL (+FasL in FIGS. 6 to 8); and one in which the construct did not include a gene encoding Fas ligand (−FasL in FIG. 6 to 8). The cells were cultured and tested for their capacity to resist FasL-induced cell death.

TABLE 4

| Construct | | |
|---|---|---|
| no. | Construct | Expressing |
| 1 | Fmc63 | CAR alone |
| 2 | Fmc63 + FasADD | CAR and Fas receptor with |
| | | a truncated death domain |
| 3 | Fmc63 + Fas-DcR2 | CAR and FLBR comprising |
| | | Fas ectodomain and DcR2 |
| | | endodomain |
| 4 | Fmc63 + Fas-41BB | CAR and FLBR comprising |
| | | Fas ectodomain and 41BB |
| | | endodomain |
| 5 | Fmc63 + Fas-OX40 | CAR and FLBR comprising |
| | | Fas ectodomain and OX40 |
| | | endodomain |
| 6 | Fmc63 + Fas-GITR | CAR and FLBR comprising |
| | | Fas ectodomain and GITR |
| | | endodomain |
| 7 | Fmc63 + Fas-CD30 | CAR and FLBR comprising |
| | | Fas ectodomain and CD30 |
| | | endodomain |
| 8 | Fmc63 + DcR3-CD8stk | CAR and FLBR comprising |
| | | Membrane bound DcR3 |
| | | (bound with a CD8 t |
| | | ransmembrane stalk) |
| 9 | Fmc63 + DcR3 | CAR and soluble DcR3 |

In more detail, freshly isolated primary human peripheral blood mononuclear cells (PBMCs) were activated with anti-CD3 and anti-CD28 antibodies (0.5 μg/ml) for 24 hours. IL-2 (100 IU/ml) was then added to the PBMCs for another 24 hours. Of the activated PBMCs, 300,000 were transduced in a 24-well plate with crude retroviral supernatant containing the gene(s) of interest.

The PBMCs were spun for 40 minutes at 1000 g at which point the cells were transferred to an incubator to be cultured for three days. Cells were then resuspended by pipetting and 100 μl was transferred to a 96-well plate. Cell survival was analysed on the flow cytometer by counting the number of surviving PBMCs.

Absolute cell numbers were normalised to PBMCs engineered to express Fmc63 alone.

The results for construct numbers 1 to 8 are shown in FIG. 6. It is clear to see that cells expressing a CAR together with Fas-DcR2, Fas-GITR, Fas-CD30 or membrane-bound DcR3 are far superior in resisting cell death than cells co-expressing a CAR with Fas-41 BB, FasOX40 or FasΔDD.

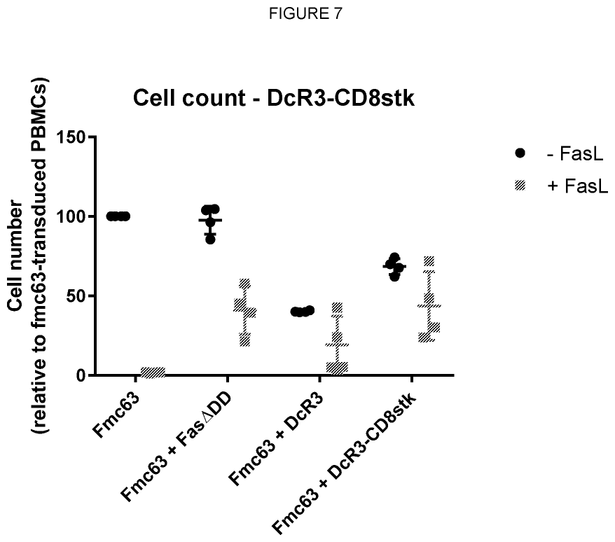

Similarly, FIG. 7 shows increased cell survival for cells expressing membrane-bound DcR3 of the present invention rather than equivalent cells expressing soluble DcR3 (comparing constructs 8 and 9).

The above methodology was repeated for constructs 1 to 7 but this time the construct also expressed the sort-suicide gene, RQR8, which is described in WO2013/153391. RQR8 to was used as a marker for transduced cells (RQR8 positive cells). It was found that cells expressing one of the FLBRs: Fas-DcR2, Fas-CD30 or Fas-GITR have a higher transduced cell number than cells expressing of Fas-ΔDD, Fas-OX40 or Fas-41BB.

### Example 2—In Vitro Testing of FasL-Binding Receptors (FLBRs) with Immobilised Fas Ligand

The panel of cells described in Example 1 which were not transduced to express FasL were tested for their capacity to resist FasL-induced cell death using immobilised soluble Fas ligand. PBMCs were transduced retroviral vectors expressing constructs 2, 3, 5 and 6 in Table 4 above, i.e. CAR+FasΔDD, Fas-DcR2, Fas-GITR and Fas-OX40.

Soluble Fas ligand (2.5 μg) was immobilised onto a 96-well plate overnight at 4° C. The Fas ligand immobilised plate was washed four times with PBS. 50,000 transduced PBMCs were added to the wells with immobilised Fas ligand and were cultured for five days in an incubator. Cells were spun by centrifugation, stained for CD3 and CD34 and cells were analysed by flow cytometry. CountBright™ absolute counting beads (Thermo Fisher) were used to determine the absolute number of surviving PBMCs. Absolute cells numbers were normalised to PBMCs transduced with Fmc63 alone in the absence of Fas ligand.

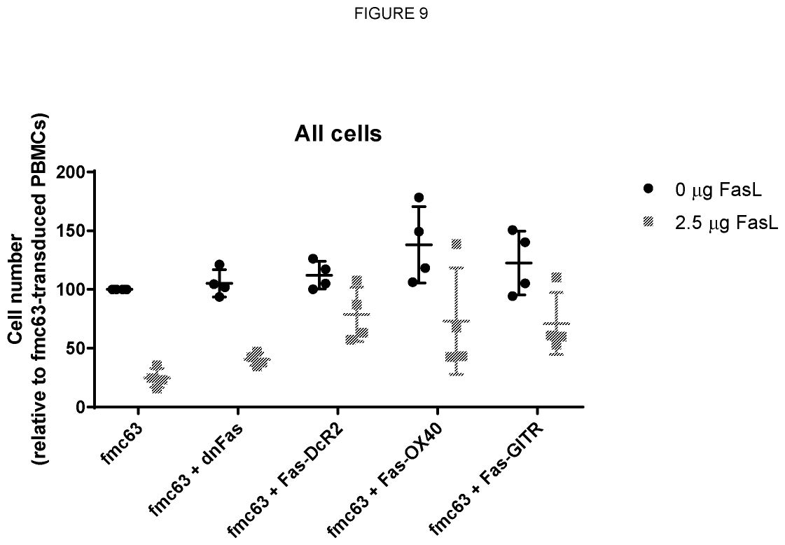

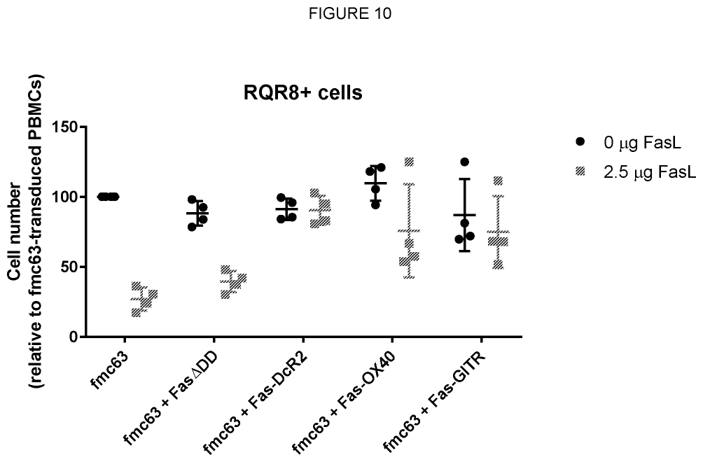

The results are shown in FIGS. 9 and 10. An increased cell count was seen for the cells expressing Fas-DcR2 and Fas-GITR cultured with immobilised FasL than was seen for cells expressing Fas-OX40 or dnFas.

### Example 3—In Vitro Testing of FasL-Binding Receptors (FLBRs) with Immobilised Fas Ligand

A panel of cells was created expressing the constructs listed in Table 5. All cells expressed an anti-CD19 CAR (Fmc63). The cells were tested for their capacity to resist FasL-induced cell death using immobilised soluble Fas ligand. Cells were tested in the presence or absence of an anti-Fmc63 anti-idiotype antibody.

TABLE 5

| Construct no. | Construct | Expressing |
|---|---|---|
| 10 | Fmc63-CD3z | CAR alone |
| 11 | Fmc63-CD3z + | CAR and FLBR comprising Fas |
| | Fas-XEDAR | ectodomain and XEDAR |
| | | endodomain |
| 12 | Fmc63-CD3z + | CAR and Fas receptor with a |
| | FasADD | truncated death domain |

PBMCs were transduced with retroviral vectors expressing the constructs as described in Example 1.

Soluble Fas ligand (2.5 μg) was either immobilised alone or in combination with 1 μg anti-Fmc63 Ab on to a 96-well plate overnight at 4° C. PBS was added to separate wells as a control. The Fas ligand/anti-Fmc63 Ab immobilised plate was washed four times with PBS. Transduced PBMCs (50,000) were added to the wells, and were cultured for five days in an incubator.

At the point of seeding cells into the 96-well plate (day 0), cells were spun by centrifugation, stained for CD3 and CD34 (to detect RQR8 and thus transduced cells) and analysed by flow cytometry to determine the percentage of cells expressing RQR8. CountBright™ absolute counting beads were used to determine the absolute number of PBMCs.

At day five of incubation, cells were spun by centrifugation, 100 μl supernatant was removed for cytokine analysis, cells were stained for CD3 and CD34 and were then analysed by flow cytometry to determine the percentage of cells expressing RQR8. CountBright™ absolute counting beads were used to determine the absolute number of surviving PBMCs. Absolute cell numbers at day five incubation were made relative to the day 0 analysis to measure proliferation fold difference. Absolute cell numbers were normalised to PBMCs cultured on PBS-treated wells.

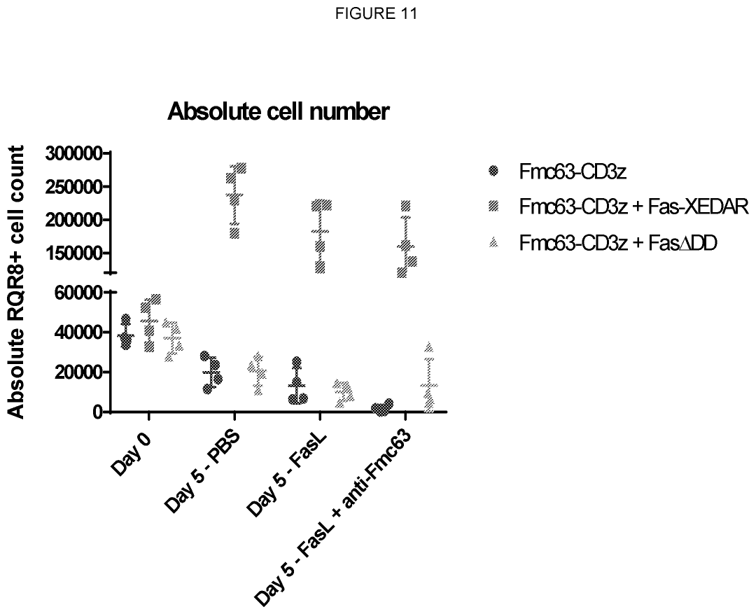

The results are shown in FIGS. 11 to 16.

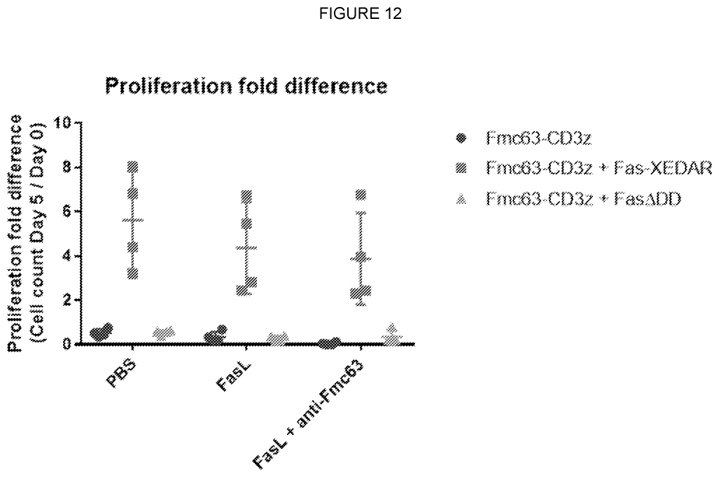

The expression of Fas-XEDAR induces constitutive proliferation of PBMCs in the absence of CAR stimulation and is able to prevent FasL-induced cell death (FIGS. 11 and 12).

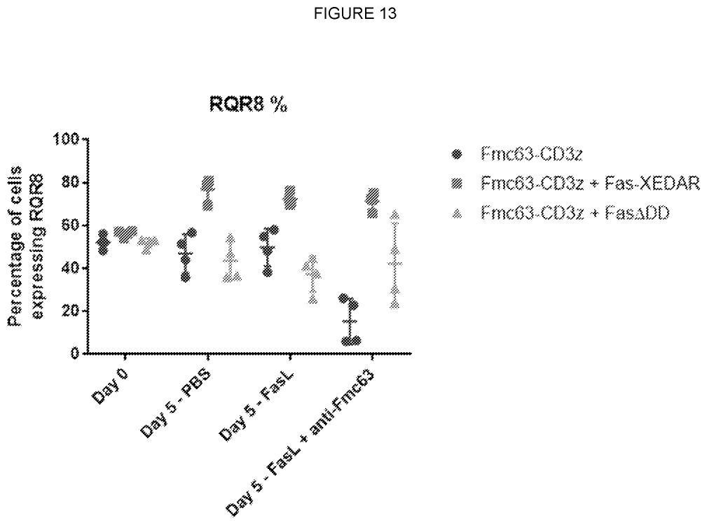

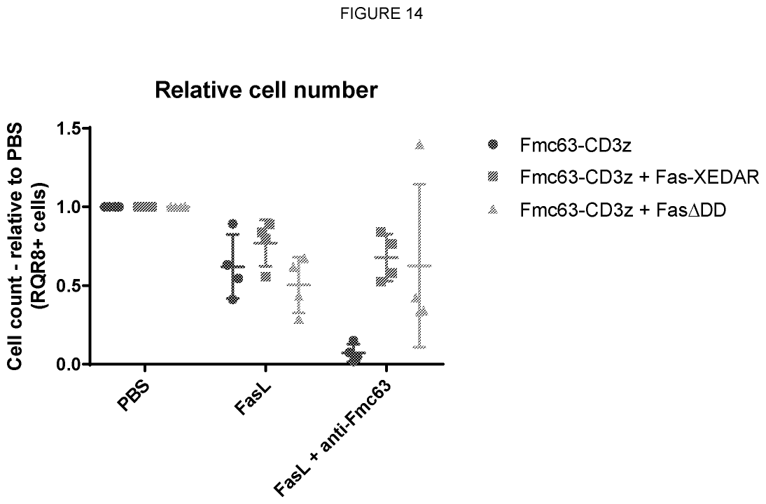

Fmc63-CD3z transduced cells are susceptible to FasL-mediated cell death. This is particularly apparent when transduced cells are co-incubated with FasL the anti-fmc63 idiotype Ab (FIG. 13, blue circles). Cells expressing Fas-XEDAR cells show greater resistance to death induced by FasL than cell expressing dominant negative Fas (i.e. Fas with a truncated death domain (FasΔDD) as shown in FIGS. 13 and 14. The cell population transduced with the vector expressing Fas-XEDAR became enriched over time for transduced cells (FIG. 13).

After the five-day incubation of transduced PBMCs on plates with either immobilised Fas ligand alone or Fas ligand/anti-Fmc63, cells were spun and 100 μl supernatant was removed for cytokine analysis. Cytokine concentrations of the supernatants were measured using ELISA MAX™ Deluxe Set Human IFN-γ (BioLegend) and ELISA MAX™ Deluxe Set Human IL-2 (BioLegend) according to the manufacturer's instructions.

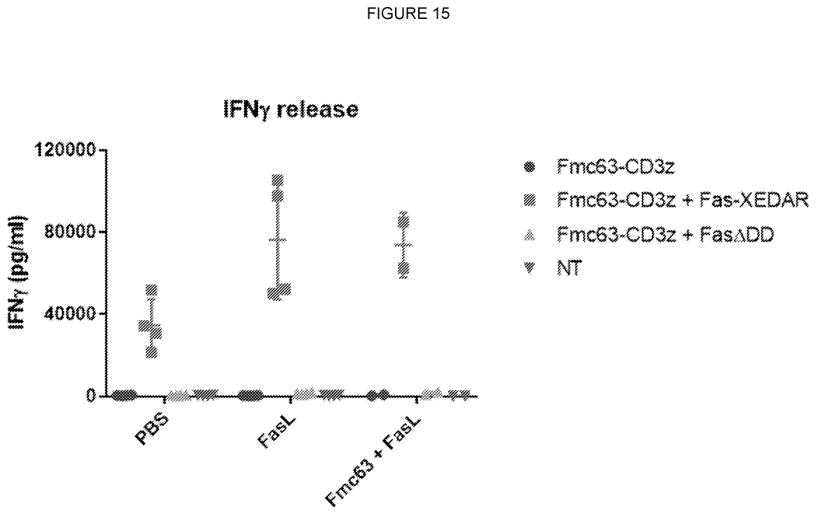

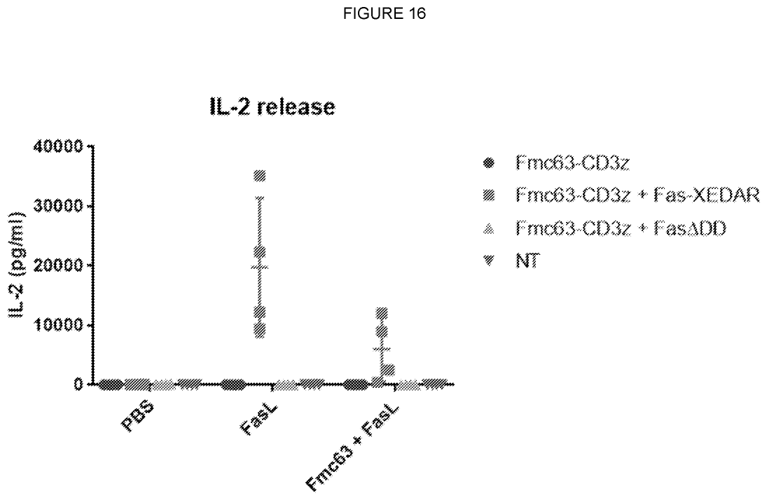

Cells expressing Fas-XEDAR showed a significantly higher release of IL2 and interferon gamma that cells expressing truncated Fas (FasΔDD) (FIGS. 15 and 16. The increase in IFNγ release was observed even in absence of immobilised Fas ligand incubation (FIG. 15).

### Example 4—Further In Vitro Screening of TNFR FasL-Binding Receptors (FLBRs) with Immobilised Fas Ligand

A panel of cells was created expressing the constructs listed in Table 6. All cells expressed an anti-CD19 CAR (Fmc63-CD3z). The cells were tested for their capacity to resist FasL-induced cell death using immobilised soluble Fas ligand.

TABLE 6

| Construct | | |
|---|---|---|
| no. | Construct | Expressing |
| 10 | Fmc63-CD3z | CAR alone |
| 12 | Fmc63-CD3z + | CAR and Fas receptor with |
| | FasADD | a truncated death domain |
| 13 | Fmc63-CD3z + | CAR and FLBR comprising |
| | Fas-HVEM | Fas ectodomain and HVEM |
| | | endodomain |
| 14 | Fmc63-CD3z + | CAR and FLBR comprising |
| | Fas-CD27 | Fas ectodomain and CD27 |
| | | endodomain |
| 15 | Fmc63-CD3z + | CAR and FLBR comprising |
| | Fas-41 BB | Fas ectodomain and 41 BB |
| | | endodomain |
| 16 | Fmc63-CD3z + | CAR and FLBR comprising |
| | Fas-CD40 | Fas ectodomain and CD40 |
| | | endodomain |
| 17 | Fmc63-CD3z + | CAR and FLBR comprising |
| | Fas-BAFF-R | Fas ectodomain and BAFF-R |
| | | endodomain |
| 18 | Fmc63-CD3z + | CAR and FLBR comprising |
| | Fas-BCMA | Fas ectodomain and BCMA |
| | | endodomain |
| 19 | Fmc63-CD3z + | CAR and FLBR comprising |
| | Fas-Fn14 | Fas ectodomain and Fn14 |
| | | endodomain |

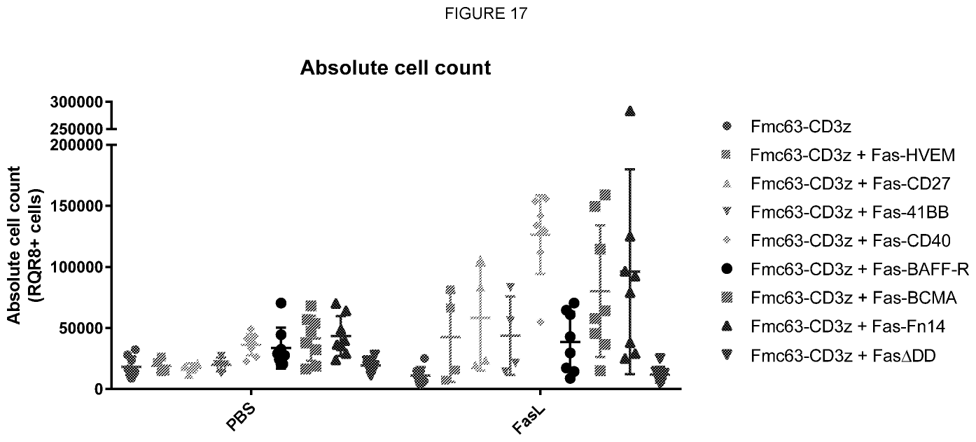

The methodology described in Example 2 was repeated using immobilised soluble Fas ligand to induce cell death. The results for are shown in FIGS. 17-20.

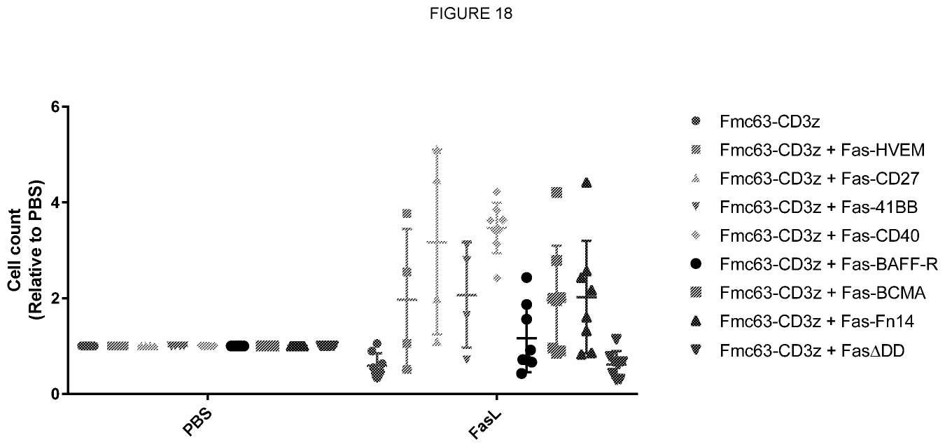

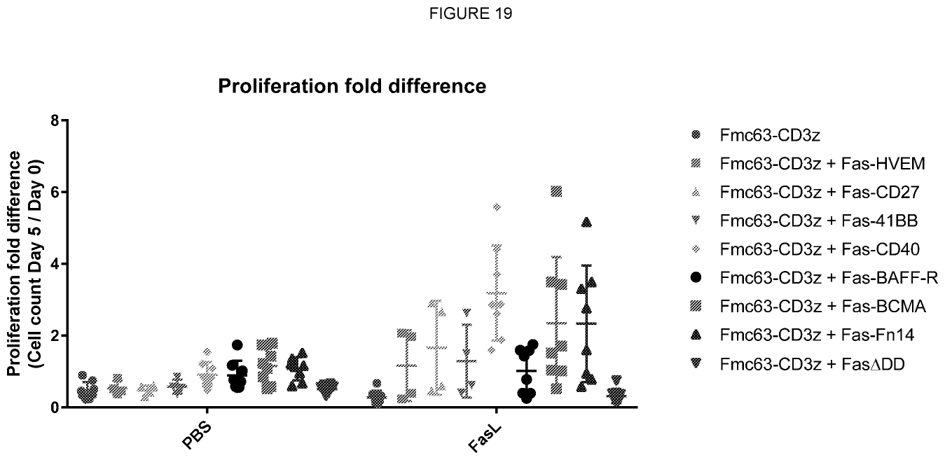

The expression of Fas-TNFR chimeras was shown to prevent FasL-induced cell death and also induce proliferation of PBMCs upon FasL-engagement. In particular, cells expressing Fas-CD27, Fas-CD40, Fas-BCMA and Fas-Fn14 showed the highest average cell count (FIG. 17). FIG. 18 shows the absolute cell count of transduced PBMCs cultured in the presence of immobilised FasL relative to transduced PBMCs cultures in the absence of FasL, confirming that expression of each of the Fas-TNFRs screened induced proliferation. The proliferation fold difference compared to the day 0 analysis (FIG. 19) showed that expression of Fas-CD27, Fas-CD40, Fas-BCMA and Fas-Fn14 induces greater proliferation of transduced PBMCs than expression of Fas-41 BB and FasΔDD.

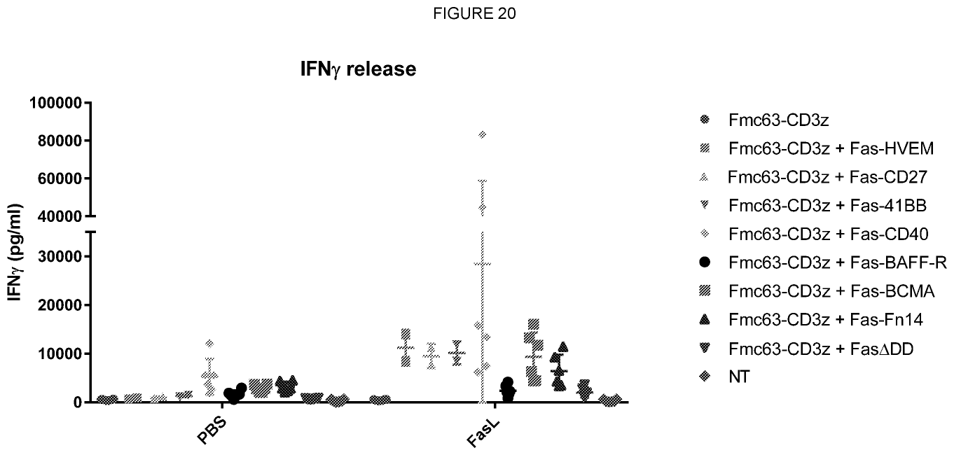

After the five-day incubation of transduced PBMCs with Fas ligand, cells were spun and 100 μl supernatant was removed for cytokine analysis. Cytokine concentrations of the supernatants were measured using ELISA MAX™ Deluxe Set Human IFN-γ (BioLegend) according to the manufacturer's instructions. As shown in FIG. 20, cells expressing Fas-CD40 showed a significantly higher release of interferon gamma than cells expressing FasΔDD. An increase in IFNγ release was observed for cells expressing the Fas-CD40 FLBR even in the absence of exposure to immobilised FasL. Cells expressing the FLBRs Fas-CD27, Fas-CD40, Fas-BCMA and Fas-Fn14 also showed a higher interferon gamma release (pg/ml) when incubated with immobilised Fas ligand when compared to that of the FasΔDD construct.

### Example 5—Investigating the Cytotoxic Capacity of Cells Co-Expressing an Anti-GD2 CAR and a TNFR FasL-Binding Receptor (FLBR) in a Restimulation Assay

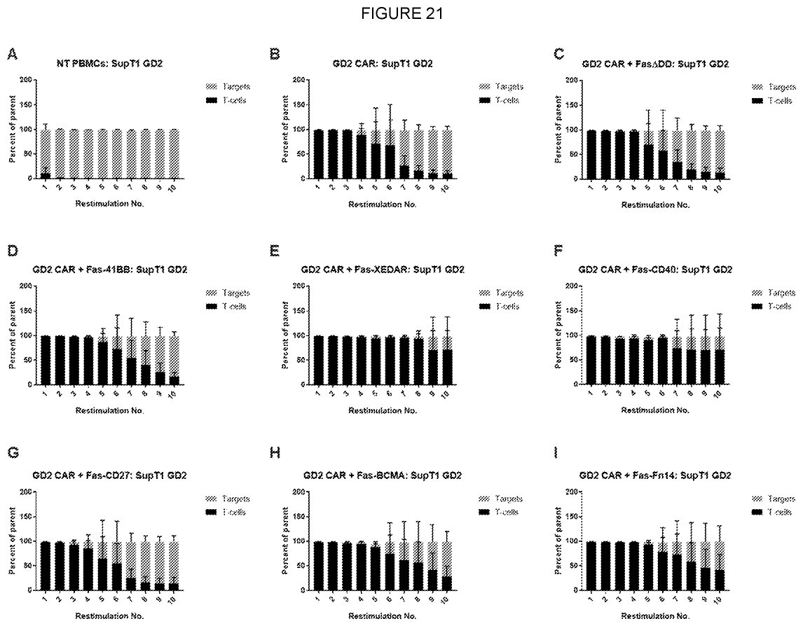

To evaluate the cytotoxic advantages of Fas-TNFR chimeras in the context of a CAR, transduced PBMCs were subject to sequential rounds of re-stimulation (FIG. 21). PBMCs were transduced to express a GD2 targeting CAR either by itself or co-expressed, via a 2A self-cleaving peptide, with either truncated Fas (FasΔDD), Fas-41 BB, Fas-XEDAR, Fas-CD40, Fas-CD27, Fas-BCMA or Fas-Fn14. Non-transduced (NT) and transduced PBMCs were co-cultured with SupT1 target cells expressing GD2 at a 1:1 effector to target ratio and cultured for four days at which point the percentage of remaining viable target cells were quantified. Every 3 or 4 days CAR-T cells were re-stimulated with 0.5×10⁵ SupT1 GD2 cells/well. GD2 CAR-T cells co-expressing Fas-XEDAR, Fas-CD40, Fas-BCMA and Fas-Fn14 (FIGS. 21 E, F, H and I) significantly outperformed control GD2 CAR-T cells and GD2 CAR-T cells co-expressing FasΔDD, Fas-41BB and Fas-CD27 (FIGS. 21 B, C, D and G) when measuring their ability to maintain cytotoxicity and expansion after repeated encounters with GD2-positive SupT1 tumour cells during in vitro sequential co-culture killing assays. Both control GD2 CAR-T cells and GD2 CAR-T cells co-expressing FasΔDD, Fas-41BB or Fas-CD27 started failing to clear the targets by the 5th re-stimulation, losing their ability to expand and eliminate GD2-positive tumour cells. In contrast, GD2 CAR-T cells co-expressing Fas-XEDAR, Fas-CD40, Fas-BCMA and Fas-Fn14 continued responding to antigen stimulation for much longer and were consequently superior at clearing target cells.

Cell Culture and Reagents

All cell lines and primary T cells used in the experiments were cultured in RPMI 1640 medium (Lonza) supplemented with 10% fetal bovine serum (FBS, Biosera) and 1% L-Glutamine (GlutaMAX, Gibco). SupT1 cells were purchased from the ATCC. T cells were generated from PBMCs obtained from National Health Service Blood and Transplant (NHSBT; Colindale, UK). Transduced T cells were cultured in the same medium as stated before, with the addition of interleukin-2 (IL-2) at 100 U/mL CountBright™ absolute counting beads (Thermo Fisher) were used to determine the absolute number of surviving PBMCs. Cytokine concentrations were measured using ELISA MAX™ Deluxe Set Human IFN-γ (BioLegend; 430104) and ELISA MAX™ Deluxe Set Human IL-2 (BioLegend; 431804) according to the manufacturer's instructions.

Transduction

The retrovirus was produced by transient transfection of 293T cells using GeneJuice (Millipore), with a plasmid encoding for gag-pol (pEQ-Pam3-E36), a plasmid encoding for the RD114 envelope (RDF37), and the desired retroviral transfer vector plasmid. Transduction was performed using Retronectin (Takara) as described previously. The transduction efficiency for the different constructs was assessed by flow cytometry based on the expression of RQR8 staining, performed using the QBEND/10 mAb. Flow cytometry analysis was performed using the MACSQuant Analyzer 10 (Miltenyi). Flow sorting was performed using a BD FACS.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which comprises;
(a) a chimeric antigen receptor (CAR) or a transgenic T-cell receptor (TCR); and
(b) a FasL-binding receptor (FLBR) comprising;
a Fas ectodomain and an endodomain from CD40 or CD27.

2. (canceled)

3. A cell according to claim 1, wherein the FLBR comprises a Fas ectodomain and a CD40 endodomain.

4. A cell according to claim 3, wherein the CD40 endodomain comprises the sequence shown as SEQ ID No. 4.

5-9. (canceled)

10. A cell according to claim 1 wherein the FLBR is selected from Fas-CD27 (SEQ ID NO: 39) and Fas-CD40 (SEQ ID NO: 41).

11. A FasL-binding receptor (FLBR) comprising a Fas ectodomain and an endodomain from CD40 or CD27.

12. A FasL-binding receptor (FLBR) according to claim 11, comprising a Fas ectodomain and a CD40 endodomain.

13. A nucleic acid sequence encoding an FLBR according to claim 11.

14. A nucleic acid construct which comprises:
(a) a first nucleic acid sequence which encodes a chimeric antigen receptor (CAR) or a transgenic T-cell receptor (TCR); and
(b) a second nucleic acid sequence which encodes a FasL-binding receptor (FLBR) according to claim 11.

15. A nucleic acid construct according to claim 14 wherein the first and second nucleic acid sequences are separated by a co-expression site.

16. (canceled)

17. A vector which comprises a nucleic acid sequence according to claim 13.

18. A kit of vectors which comprises:
(a) a first vector which comprises a nucleic acid sequence which encodes a chimeric antigen receptor (CAR) or a transgenic T-cell receptor (TCR); and
(b) a second vector which comprises a nucleic acid sequence which encodes a FasL-binding receptor (FLBR) according to claim 11.

19. A pharmaceutical composition which comprises a plurality of cells according to claim 1.

20. (canceled)

21. A method for treating and/or preventing a disease, which comprises the step of administering a pharmaceutical composition according to claim 19 to a subject in need thereof.

22. A method according to claim 21, which comprises the following steps:
(i) isolation of a cell containing sample;
(ii) transduction or transfection of the cell with a nucleic acid sequence encoding a FasL-binding receptor (FLBR) comprising a Fas ectodomain and an endodomain from CD40 or CD27; and
(iii) administering the cells from (ii) to a subject.

23. The method according to claim 22 wherein the cell is autologous.

24. The method according to claim 21 wherein the cell is allogenic.

25. (canceled)

26. A method according to claim 22, wherein the disease is cancer.

27. A method for making a cell according to claim 1, which comprises the step of introducing: a nucleic acid sequence encoding a FasL-binding receptor (FLBR) comprising a Fas ectodomain and an endodomain from CD40 or CD27 into the cell ex vivo.

28. A method according to claim 27, wherein the cell is from a sample isolated from a subject.


SEQUENCE LISTING (55 sequences)

SEQ ID NO: 1 - protein, 58 aa, Homo sapiens
QLGLHIWQLRSQCMWPRETQLLLEVPPSTEDARSCQFPEEERGERSAEEKGRLGDLWV

SEQ ID NO: 2 - protein, 188 aa, Homo sapiens
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 3 - protein, 169 aa, Homo sapiens
TSNTKCKEEGSRSNLGWLCLLLLPIPLIVWVLYCKQFFNRHCQRGGLLQFEADKTAKEESLFPVPPSKETSAESQVSENIFQTQPLNPILEDDCSSTSGFPTQESFTMASCTSESHSHWVHSPIECTELDLQKFSSSASYTGAETLGGNTVESTGDRLELNVPFEVPSP

SEQ ID NO: 4 - protein, 62 aa, Homo sapiens
KKVAKKPTNKAPHPKQEPQEINFPDDLPGSNTAAPVQETLHGCQPVTQEDGKESRISVQERQ

SEQ ID NO: 5 - protein, 48 aa, Homo sapiens
QRRKYRSNKGESPVEPAEPCHYSCPREEEGSTIPIQEDYRKPEPACSP

SEQ ID NO: 6 - protein, 107 aa, Homo sapiens
RKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 7 - protein, 28 aa, Homo sapiens
RRCRRREKFTTPIEETGGEGCPAVALIQ

SEQ ID NO: 8 - protein, 154 aa, Homo sapiens
RKKFISYLKGICSGGGGGPERVHRVLFRRRSCPSRVPGAEDNARNETLSNRYLQPTQVSEQEIQGQELAELTGVTVESPEEPQRLLEQAEAEGCQRRRLLVPVNDADSADISTLLDASATLEEGHAKETIQDQLVGSEKLFYEEDEAGSATSCL

SEQ ID NO: 9 - protein, 271 aa, Artificial Sequence
  FLBR DcR3 domain sequence
VAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 10 - protein, 270 aa, Artificial Sequence
  FLBR sequence, mutant DcR3
VAETPTYPWRDAETGERLVCAQCPPGTFVQRPCRRDSPTTCGPCPPRHYTQFWNLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLALAARLTELLGAQDGALLVRLLQALRVARMPGLERSVRERFLPVH

SEQ ID NO: 11 - protein, 46 aa, Artificial Sequence
  spacer sequence, CD8stk
PTTTPAPRPPTPAPTIASQPLSLRPEACRPAAGGAVHTRGLDFACD

SEQ ID NO: 12 - protein, 15 aa, Artificial Sequence
  spacer sequence, Hinge
EPKSCDKTHTCPPCP

SEQ ID NO: 13 - protein, 51 aa, Artificial Sequence
  spacer sequence, COMP
AGSDLGPQMLRELQETNAALQDVRELLRQQVREITFLKNTVMECDACGSGK

SEQ ID NO: 14 - protein, 110 aa, Artificial Sequence
  spacer sequence, CH1-Hinge
KGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 15 - protein, 327 aa, Artificial Sequence
  spacer sequence, CH1-Hinge-CH2-CH3
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 107 aa, Artificial Sequence [sequence omitted]

SEQ ID NO: 17 - protein, 41 aa, Artificial Sequence
  spacer sequence, CD28 Stalk
AKIEVMYPPPYLDNEKSNGTIIHVKGKHLCPSPLFPGPSKP

SEQ ID NO: 18 - protein, 4 aa, Artificial Sequence
  polar anchor sequence
RKKR

SEQ ID NO: 19 - protein, 50 aa, Artificial Sequence
  rigid linker with truncations
LEAEAAAKEAAAKEAAAKEAAAKALEAEAAAKEAAAKEAAAKEAAAKALE

SEQ ID NO: 20 - protein, 17 aa, Homo sapiens
LGWLCLLLLPIPLIVWV

SEQ ID NO: 21 - protein, 21 aa, Artificial Sequence
  DcR2 transmembrane domain
YLIIIVVLVIILAVVVVGFSC

SEQ ID NO: 22 - protein, 21 aa, Artificial Sequence
  GITR transmembrane domain
LGWLTVVLLAVAACVLLLTSA

SEQ ID NO: 23 - protein, 21 aa, Artificial Sequence
  CD30 transmembrane domain
PVLFWVILVLVVVVGSSAFLL

SEQ ID NO: 24 - protein, 34 aa, Artificial Sequence
  CD8 transmembrane domain
APTIASQPLSLRPEACRPAAGGAVHTRGLDFACD

SEQ ID NO: 25 - protein, 21 aa, Artificial Sequence
  CD28 transmembrane domain
FLFVLLGVGSMGVAAIVWGAW

SEQ ID NO: 26 - protein, 27 aa, Artificial Sequence
  4-1BB transmembrane domain
IISFFLALTSTALLFLLFFLTLRFSVV

SEQ ID NO: 27 - protein, 21 aa, Artificial Sequence
  DR3 transmembrane domain
MFWVQVLLAGLVVPLLLGATL

SEQ ID NO: 28 - protein, 21 aa, Artificial Sequence
  OX40 transmembrane domain
VAAILGLGLVLGLLGPLAILL

SEQ ID NO: 29 - protein, 21 aa, Artificial Sequence
  CD70 transmembrane domain
VLRAALVPLVAGLVICLVVCI

SEQ ID NO: 30 - protein, 22 aa, Artificial Sequence
  CD40 transmembrane domain
ALVVIPIIFGILFAILLVLVFI

SEQ ID NO: 31 - protein, 21 aa, Artificial Sequence
  XEDAR transmembrane domain
LVALVSSLLVVFTLAFLGLFF

SEQ ID NO: 32 - protein, 21 aa, Artificial Sequence
  Fn14 transmembrane domain
ILGGALSLTFVLGLLSGFLVW

SEQ ID NO: 33 - protein, 23 aa, Artificial Sequence
  BCMA transmembrane domain
ILWTCLGLSLIISLAVFVLMFLL

SEQ ID NO: 34 - protein, 21 aa, Artificial Sequence
  CD27 transmembrane domain
ILVIFSGMFLVFTLAGALFLH

SEQ ID NO: 35 - protein, 338 aa, Artificial Sequence
  FasL-binding receptor (FLBR) sequence, human Fas-Dcr2
MGWSCIILFLVATATGVHSQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVRKKFISYLKGICSGGGGGPERVHRVLFRRRSCPSRVPGAEDNARNETLSNRYLQPTQVSEQEIQGQELAELTGVTVESPEEPQRLLEQAEAEGCQRRRLLVPVNDADSADISTLLDASATLEEGHAKETIQDQLVGSEKLFYEEDEAGSATSCL

SEQ ID NO: 36 - protein, 244 aa, Artificial Sequence
  FLBR sequence, human Fas-GITR
MGWSCIILFLVATATGVHSQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVRKQLGLHIWQLRSQCMWPRETQLLLEVPPSTEDARSCQFPEEERGERSAEEKGRLGDLWV

SEQ ID NO: 37 - protein, 374 aa, Artificial Sequence
  FLBR sequence, human Fas-CD30
MGWSCIILFLVATATGVHSQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVRKHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 38 - protein, 322 aa, Artificial Sequence
  FLBR sequence, human Fas-XEDAR
MGWSCIILFLVATATGVHSQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVLYCKQFFNRHCQRGGLLQFEADKTAKEESLFPVPPSKETSAESQVSENIFQTQPLNPILEDDCSSTSGFPTQESFTMASCTSESHSHWVHSPIECTELDLQKFSSSASYTGAETLGGNTVESTGDRLELNVPFEVPSP

SEQ ID NO: 39 - protein, 232 aa, Artificial Sequence
  FLBR sequence, human Fas-CD27
MGWSCIILFLVATATGVHSQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVQRRKYRSNKGESPVEPAEPCHYSCPREEEGSTIPIQEDYRKPEPACSP

SEQ ID NO: 40 - protein, 291 aa, Artificial Sequence
  FLBR sequence, human Fas-BCMA
MGWSCIILFLVATATGVHSQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 41 - protein, 246 aa, Artificial Sequence
  FLBR sequence, human Fas-CD40
MGWSCIILFLVATATGVHSQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKKVAKKPTNKAPHPKQEPQEINFPDDLPGSNTAAPVQETLHGCQPVTQEDGKESRISVQERQ

SEQ ID NO: 42 - protein, 212 aa, Artificial Sequence
  FLBR sequence, human Fas-Fn14
MGWSCIILFLVATATGVHSQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVRRCRRREKFTTPIEETGGEGCPAVALIQ

SEQ ID NO: 43 - protein, 394 aa, Artificial Sequence
  complete FLBR sequence, human DcR3-CD8 stalk/TM/rigid linker
METDTLILWVLLLLVPGSTGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVHSDPTTTPAPRPPTPAPTIASQPLSLRPEACRPAAGGAVHTRGLDFACDIYIWAPLAGTCGVLLLSLVITLYCRKKRSLEAEAAAKEAAAKEAAAKEAAAKALE

SEQ ID NO: 44 - protein, 394 aa, Artificial Sequence
  complete FLBR sequence, human mutant DcR3-CD8 stalk/TM/rigid linker
METDTLILWVLLLLVPGSTGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLALAARLTELLGAQDGALLVRLLQALRVARMPGLERSVRERFLPVHSDPTTTPAPRPPTPAPTIASQPLSLRPEACRPAAGGAVHTRGLDFACDIYIWAPLAGTCGVLLLSLVITLYCRKKRSLEAEAAAKEAAAKEAAAKEAAAKALE

SEQ ID NO: 45 - protein, 112 aa, Artificial Sequence
  CD3-zeta endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 368 aa, Artificial Sequence
  4-1BB and CD3-zeta endodomains
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 47 - protein, 152 aa, Artificial Sequence
  CD28 and CD3-zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 188 aa, Artificial Sequence
  CD28, OX40 and CD3-zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 50 - protein, 148 aa, Homo sapiens
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 51 - protein, 144 aa, Homo sapiens
KRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITDSENSNFRNEIQSLV

SEQ ID NO: 52 - protein, 27 aa, Artificial Sequence
  CD28 transmembrane domain (TM) domain
FWVLVVVGGVLACYSLLVTVAFIIFWV

SEQ ID NO: 53 - protein, 21 aa, Artificial Sequence
  CD8 TM domain
IYIWAPLAGTCGVLLLSLVIT

SEQ ID NO: 54 - protein, 20 aa, Artificial Sequence
  cleavage site, 2A-like sequence
RAEGRGSLLTCGDVEENPGP

SEQ ID NO: 55 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX